SEQUENCE LISTING (5 sequences)

SEQ ID NO: 1 - DNA, 4221 bp, Homo sapiens
gtcggccgtcccctttaatttttaaatacacggtcccctcttttctctggggggggcaagcaagaaatcaaagaaggaggagacaagccgtcaattttctccaaaacaaaccccaccgggcaatttggtctcggggtagggggagacggggtgattgcaaattattccaggacgagatccagttctccagcgggaaaggggcaaaggaacgccgcgcgttggaagggccagggtacgcagctccccttgcagcgcccgcaggacccccgcaagctcgtgccggcgaaatcggagaccgccgatctgtcctcgttctctcctgcacgtctggctgcattcggaggaagacctggggcgcgagcgagcggcgacagcatgagcctgtgctgacctccgcgcggcgggccgagcccagggctttgtcgcggtacctgcgcccagcccgcgccgcaactctgtgcccagcttttgcaatcttttgttgcagcgctgaccgcaccaagttaaatgctcccttgcaatttttcttttttttgtttgtttgtttaatttttggagagctcgcgatcttggaaaagcctcagacgccatctacagttaaaacgtaggtaactgccctctcccgcaccccccccttacacgccccccaccctttccaccaaaaaaagggggtgcagcgcggattctggctgccgtgcgtcgccagccggtagacccgtgcttgtttcctttctctttttgtttggcttctaacgcgttgggactgagtcgccgccgtgagctccccgaagactgcacaaactaccgcgggctcctccgccccgtctgcgattcggaagccggcctgggggtcgcgtcgggagccctgcgctgcagctccgcaccttagcagcccgggtactcatccagatccacgccggggacacacacacagagtaactaaaagtgcggcgattctgcacatcgccgactgctttggggtaacaaaaagacccgagttgcctgccgaccgaggacccccgggagccgggctcggagcagacgaggtatccggcggcgcccatttgggggcttctaactctttctccacgcagcccctcttctgtcccctcccctctcgctcccttttaaaatcagtggcaccgaggcgcctgcagccgcactcgccagcgactcatctctccagcgggtttttttttgtttgtcgtgtgcgatcctcacactcatgaacatacacaggtctacccccatcacaatagcgagatatgggagatcgcggaacaaaacccaggatttcgaagagttgtcgtctataaggtccgcggagcccagccagagtttcagcccgaacctcggctccccgagcccgcccgagactccgaacttgtcgcattgcgtttcttgtatcgggaaatacttattgttggaacctctggagggagaccacgtttttcgtgccgtgcatctgcacagcggagaggagctggtgtgcaaggtgtttgatatcagctgctaccaggaatccctggcaccgtgcttttgcctgtctgctcatagtaacatcaaccaaatcactgaaattatcctgggtgagaccaaagcctatgtgttctttgagcgaagctatggggacatgcattccttcgtccgcacctgcaagaagctgagagaggaggaggcagccagactgttctaccagattgcctcggcagtggcccactgccatgacggggggctggtgctgcgggacctcaagctgcggaaattcatctttaaggacgaagagaggactcgggtcaagctggaaagcctggaagacgcctacattctgcggggagatgatgattccctctccgacaagcatggctgcccggcttacgtaagcccagagatcttgaacaccagtggcagctactcgggcaaagcagccgacgtgtggagcctgggggtgatgctgtacaccatgttggtggggcggtaccctttccatgacattgaacccagctccctcttcagcaagatccggcgtggccagttcaacattccagagactctgtcgcccaaggccaagtgcctcatccgaagcattctgcgtcgggagccctcagagcggctgacctcgcaggaaattctggaccatccttggttttctacagattttagcgtctcgaattcagcatatggtgctaaggaagtgtctgaccagctggtgccggacgtcaacatggaagagaacttggaccctttctttaactgagctcatgccccacggagacttagcaggttccaggagtgagcgagggcagcggaaaggagttcttccgggggacacgaattgcctggctgagtagcaagaaagacacactcttaagtttcttggttcagagcaggaaaaccttcaaggagctgactgaccacgtagcatgggggcaagaggcgtgggatggggattggggtgagatggatgggagcccgctggagcttgtcttccctaacatagcctgggagaccaccccttgccacttgggccacttccgcctaccccacttttcattttgttccaaaatagttgcagatcctgacagaatcaaaactctctgcctcaaacacacatcctggcatcgcactgttagcatttaacttcttgttaggattcagggaaggaacagttggccaagaattttttttcttttaaacaagccaaccacctagctggtaattaatgaggttcacttaaaaaaaaaattcggtgcacacagactgacatgaaacctgggtgctacagtaaaagaaaacaaaagtccagtttgtgtctcttaatcgctcacttcaactcatttcttctaaataaactatttaatatcctggtcaggaaatgacatgttaatgctttgctccctgaagggggaaaaaatctgtcctttaacaagctattctgttttgtgtcaattgggtccgtggcaaggaagctattaggaagtcaaacggtccaggatgcattacctgctaatccttaggtttaaagggggaaagaaaagggaagaagaaaggaaaagagaaatccaactcctttttcatgttttgcttttgaacaatgagggtttgtgtgacaggcattcctctttgctgagatgatagcaatggcctgagattttagcaagctcctggagtctgatgcttttgcagtactctgatcgcaactaaacatttgtctttgttttattagaaactagtgaaacaaagcaggttgtcccacatgtataaaatacagggcagctatttagttttctttacagagaatgatccttttaaggcttgtaaggccctctggtttggacaaaaaccctcagtagagacaagcgggaaggataattagctgaaagctatgatgatataaataaaaacagctctctatcccaatacgcacctttgtattttcaagaactcttctatttattaaggaaaatgtcacattgtgatgtattaagccagtacttcaattacgggttgacttgggatgacatattacatgctgtagttaacatttataattctttttccttgtttgagtatttctgtctctgaaataaccttttacttggcttttctagatagctttatttgatttcgagtggcaaaatgttttttattacggcttttctattgctgtatgatacagaactcttttggcataaatatttgtgttcccagtacctcacttgttcggatttgactgcctgtatatgttttgtgaaatggtcctgtttttgggtaggtgacacgtggactctagtatgtaaatgttacttgaatctgtgcttcataatagtgtgtggcatgtatgtgcagactcttggatgctttatgcctgcgcaccaggagccctgtcctcacgttcccaggagggcggcttcacccttcgtaaccaggagacaaggcggccatggatttgcccttgattctattttgctaatggaagatagaaaggagagaaggtttttttttttttttaacattctgaagatggtgctgtgtcaagaaggaccttttttttcccctctcccctattttttaagtaccttggaggaggagaggttggtgacatgcatggtggggatctatggcctctggtgctttgtcctgtatttggtttaatgtttttgtcctaatctcttcaatcaataaaattgtgcgtatttaactaaaaaaaaaaaaaaaaaa

SEQ ID NO: 2 - protein, 343 aa, Homo sapiens
MNIHRSTPITIARYGRSRNKTQDFEELSSIRSAEPSQSFSPNLGSPSPPETPNLSHCVSCIGKYLLLEPLEGDHVFRAVHLHSGEELVCKVFDISCYQESLAPCFCLSAHSNINQITEIILGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTSGSYSGKAADVWSLGVMLYTMLVGRYPFHDIEPSSLFSKIRRGQFNIPETLSPKAKCLIRSILRREPSERLTSQEILDHPWFSTDFSVSNSAYGAKEVSDQLVPDVNMEENLDPFFN

SEQ ID NO: 3 - DNA, 4198 bp, Drosophila
cacgtaagtaactattaataccgcctcgcccggggatgcgggcgtgcttgagcgccgggattggcctagggcgccgtggccatcccctttaatttttaaatacacggtcctctcctacttctgggtggcaagaaaaaaaaaataataagaaaagaagacaagccgtcgatttttatttttcctgcagagcaaaccccgccgtgcaatttggtcgcaaggcatcgggagacaagagtggtttgcaaattgtttcaaggctcaagacccagttgtgcagcggaaaaggaacgccgcgggttgacggagtccggggagcatctgctggcagctcctgcaggacccccggaaagctcctgctgctggtaccccccaggtcaccgcccgcacttcttgcttcatcggaggaggatctgggacgtgagcagcgacagcaagagctagtgctgaccgcgcgggctctgccttgggctttgtcgcggtacctgtgcctgccctactccgcgcactccctactcagcttttgcaatcttttgttgccactgctgaccgccccaagttaaaagcgcctttgcaattttttttttctgtctttttttttttttcttgagagagacagctctgcgattttttggaaaagcctcagacgccatctacagttaagacttaggtaactgccctctctagcaccccccttacacgccccccaccctttccaccaaaaagaggggggtgcagcgcggattctggctgccgcgcggcgtgagccggtagacccgagcttatttcctttttctttttgtttggtttctaacgcgtggagggcgagccggcgccgcgcgctccctgaagactgcacaaactccacgcagggcttctccgcccggtctgcggatcctcagctggggatcgctcagaagcccggcgctgcagctcctcaccccagaggcacgctcactcgtccagatccacgctgcgaacagagacccactgagtccagcgtgcggttctgcaccgcgctggcagcttctgggtaacaaaaggacccgagttgtccgcagagcgagcacccccgggagcggggctcgcagccggggaccagccctgcagcgcccatctgggggctagttcttaactcttcctccacggagccccagacgggtcccctcccttcttgatccttttaagtcggtagcaccgaggcgcctgcaccggcgcggctcatccatctctccagaggggttttttggtttgtttgtttgttttgtcgtgtgcgatcctcacactcatgaacatacacaggtctacccctatcacaatagcgagatatgggagatcgcggaacaaaacccaggatttcgaagagctgtcgtctataaggtccgctgagcccagccagagtttcagcccgaaccttaactctccgagcccgcccgagactccgaacttgtcgcattgcgtttcttgcatcgggaaatacttactgttggagcctctggagggagaccacgttttccgcgctgtgcatctgcacagcggagaggagctggtttgcaaggtgtttgagatcagctgctaccaggagtccctggccccctgcttctgcctgtctgcccatagcaacatcaaccaaatcacggaaatcctcctgggagagaccaaagcctatgtgttctttgagcgaagctatggagacatgcattcctttgtccgcacttgtaagaagctgagggaggaggaggcagcccgactgttctaccagattgcctcagctgtggcccattgccacgatggaggcctggtgctgcgtgacctcaagctgcggaaatttatcttcaaggatgaagagaggactcgtgtcaagctggagagtttggaagacgcttacattctccggggtgatgatgactcactctctgacaagcatggctgcccagcgtatgtcagcccagagatcttgaacaccagcggcagttattcgggcaaggcagcggacgtgtggagcctgggggtaatgctgtacaccatgttggtggggcgttaccctttccatgacattgagcctagttctcttttcagtaagatccgcaggggccagttcaacattccagaaactctgtctcccaaggccaagtgcctcatccgaagcatcctgcgacgggagccgtcagagcggctgacctcgcaggaaattctggaccatccttggttttctacagattttagtgtctcaaattcgggatttggtgctaaagaggcgtgtgaccagctggtgccagacgtcaacatggaggagaacttggaccctttctttaactgagctcaaggcccagggacacatagcaggtaccaggagcaagagagagccccagaaaggagttctgggacacaggtggcctggctgagaagcaagacggacattcatatttacacatttcttggttcagagaaggaatatgttctaggagctgacggaacacgtagcatgggaacaagacgtgtgggatgggggttgggttcagatggacgggagcccctcccctaagcttctcttccctggggtagcctgagagtcccccttaccagtagggctattctaccccacttttcattttgttcagaaatagttgcagatctcgatagaatccaaactcttctgcctcaaacacctaccttggcattgcactgttagcatttaacttcttgttacgattcagggaagggacaattgatcgaagattttttttttttttttggaacagaccaaccacctatgtaataattaataagattcacctaaaaataataataattcggtgcacacagactgacctgaaacctgggtgctaaactaaaagaaaacaaaagttccagttgtcgtctctcattcgcactttccaattcatttcttctaaataaacgatgtcctattctggttaggaagtaacacattaacgctttgctccctgaacggggagggggagtctgttcctccacagacatttctgttttgtatcagctggtttttgtagcaggaaactatcagaagtcaaacttccagatgtattatcacagttcaggggaagaagaaaggaaaagaagaaaaatccaactcctttctggtttttgttcttttgaaggaagagggttcacattgtagacattgctctctgctccaaattcagtgaggggctcccagagggcaggcgcctcctggagtcagatctttttgatgatgctgatctcaacgttttgtttttgctttatgggaaactagtaaaacgagacaggttgtcccatgtgtataaaatacagggcagctatttccttttctttgctaagaatgatcctttgggcttggaaaggccctctggtttgaacagaaagagtaaacgggcaataagccaaaagccaggatgatatacatacaaacagctctctgtcccaatacgcaccttgtatttattaaggaaaatgtcacattgtgatgtattaagccagtacttcaattacgggtcaacgggatgacatgttacatgctgtagtttaacatttataattttgttcccctgttttgagtatttctgtccctggaataaccttttatttggctttctctagatagccttatttgattttgagtggcaaaatgtttttccttttgtactctggcttttctattgctgtatgatacagaactcttttggcataaatatttgtgttcccagtacctcagtcgtttgggttttcctgcctgcatctgttttgtgaaatggtcctgtttttgggtaggtgacacgtggactctagtgtgtaaatgttacttgaatctgtgcttcactctagtatgtggcatgtgtgtgcggactcttggatgcttcacgcctactccactggagcccctgtccccaggaggacagcttccccactgataatcaggagaccaagctgccatggatttacccttgattctattttgataatggaagatacagagagagggtttttacattcagaagatggtgctgtggcaagaaggaccttttatcttccctctcccctgtttttaaagtcctcggtgggaggaaagattggaaacatgcatgatggggactaatggcctctggtgctttgtcctgtatttggtttaatgtttttgtcctaatctcttcaatcaataaaattgtgcgtatttaactaa

SEQ ID NO: 4 - protein, 343 aa, Murinae gen. sp.
MNIHRSTPITIARYGRSRNKTQDFEELSSIRSAEPSQSFSPNLGSPSPPETPNLSHCVSCIGKYLLLEPLEGDHVFRAVHLHSGEELVCKVFEISCYQESLAPCFCLSAHSNINQITEILLGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTSGSYSGKAADVWSLGVMLYTMLVGRYPFHDIEPSSLFSKIRRGQFNIPETLSPKAKCLIRSILRREPSERLTSQEILDHPWFSTDFSVSNSGFGAKEACDQLVPDVNMEENLDPFFN

SEQ ID NO: 5 - DNA, 20 bp, Artificial Sequence
  promoter consensus sequence
aaggtgttgcaatccccagc

(19) United States
(12) Patent Application Publication
(10) Pub. No.: US 2011/0014214 A1
(43) Pub. Date: Jan. 20, 2011

(54) Diagnostic and Therapeutic Utility of Tribbles-2 in Human Cancers

(75) Inventors: Warren S. Pear, Philadelphia, PA (US); Karen Keeshan, Philadephia, PA (US); Yiping He, Phildephia, PA (US)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(73) Assignee: The Trustees of the University of Pennsylvania

(21) Appl. No.: 12/087,949

(22) PCT Filed: Jan. 19, 2007
(86) PCT No.: PCT/US07/01638

§ 371 Date: Jun. 14, 2010

Related U.S. Application Data
(60) Provisional application No. 60/760,856, filed on Jan. 20, 2006.

(51) Int. Cl. A61K 39/395; C12Q 1/68; G01N 33/68; C12N 5/078; A61K 38/02; A61K 31/7105; A61K 31/7088; A61P 35/02

(52) U.S. Cl. 424/173.1; 435/6; 435/7.24; 435/375; 514/1.1; 514/44 A; 514/44 R; 514/19.6

(57) ABSTRACT

Provided are methods for the diagnosis and treatment of acute myelogenous leukemia. In particular, the present invention relates to the use of Trib2 polynucleotides and polypeptides for the diagnosis and treatment of acute myelogenous leukemia (AML) by assessing myeloid cells of a patient, or malignancies associated with Trib2, C/EBPαp30 or C/EBPαp42, such as AML or lung cancer, by assessing hematopoietic stem cells of the patient.

### BACKGROUND OF THE INVENTION

Acute Myeloid Leukemia (AML) is a genetically and phenotypically heterogenous disease that is characterized by a block in myeloid differentiation, and enhanced proliferation and survival (reviewed in (Kelly et al., Annu. Rev. Genomics Hum. Genet. 3:179-198 (2002)). Chromosomal translocations that target transcription factors are commonly associated with AML including core binding factor (CBF), and retinoic acid receptor alpha (RARα), resulting in fusion proteins including AML1/ETO (t[8;21]), CBFb/SMMHC (inv[16]), TEL/AML1 (t[12;21]), and PML/RARα (t[15;17]). Mutations in transcription factors themselves are also frequently associated with AML. Among the most commonly studied transcription factors in AML are PU.1, C/EBPα, AML1, and GATA-1. In addition to mutations found in these transcription factors, modulation of their transcription factor function is associated with AML disease (Rosenbauer et al., Blood 106:1519-1524 (2005)). Mutations are also found in genes in AML that confer proliferative and survival advantages to the cells including FLT3, RAS and c-Kit. Updating and extending the list of genes found perturbed in AML remains a major goal in leukemia research.

The tribbles gene (“Trib”) was first identified in Drosophila by mutations that disrupted gastrulation and oogenesis (Grosshans et al., Cell 101:523-531 (2000); Mata et al., Cell 101:511-522 (2000); Seher et al., Curr. Biol. 10:623-629 (2000)). Trib2 is a mammalian homolog of Drosophila tribbles, and there are two other mammalian counterparts, Trib1 and Trib3. All tribbles proteins closely resemble serine-threonine kinases, but are believed to be functionally dead as they contain a variant catalytic core and lack the ATP binding site of conventional kinases. The N-terminal region shows the least homology amongst Trib family members and to Drosophila tribbles (Kiss-Toth et al., Cell Signal 18:202-214 (2006)). Drosophila embryos with tribbles loss of function have low viability, with only 14% of mutant flies surviving to adulthood, and loss of homozygotes did appear to occur at a specific developmental stage.

Trib is required for the coordination of cell division with gastrulation and functions by controlling the cell cycle protein String/CDC25. Trib specifically promotes String protein turnover via the proteasome to prevent premature mitosis during gastrulation (Mata et al., supra, 2000). Mutation at a crucial lysine of the catalytic center did not prevent the premature pause in cell cycle seen by tribbles overexpression indicating that tribbles does not function as a conventional kinase (Grosshans et al., Cell 101:523-531 (2000)). Overexpression of tribbles outside the germ cells was shown to slow the cell cycle in wing imaginal disc cells (Mata et al., supra, 2000). In addition, it has been shown to be a negative regulator of slbo, the Drosophila homolog of the C/EBP family of basic region-leucine zipper transcription factors, in border cell migration during oogenesis, by specifically binding and stimulating the ubiquitin-mediated proteolysis of slbo (Rorth et al., Mol. Cell 6:23-30 (2000)).

Of the three mammalian Trib family members, most data exists for Trib3, which has been shown to function as a negative regulator of AKT in the liver in fasting conditions by directly binding and inhibiting AKT phosphorylation (Du et al., Science 300:1574-1577 (2003)). Trib3 is also a transcriptional target of the nuclear hormone receptor PPAR-α in the liver (Koo et al., Nat. Med. 10:530-534 (2004)). Both of these studies demonstrate that Trib3 affects insulin signaling in the liver. Trib3 (previously Skip3) has been shown to be upregulated in tumor cells and in hypoxic conditions. Like the Drosophila counterpart, Trib3 lacks kinase activity using traditional serine/threonine kinase substrates (Bowers et al., Oncogene 22:2823-2835 (2003)). Reports have also shown that human Trib3 is able to interact with and affect the activity of ATF4 in stressful conditions (Bowers et al., Oncogene 22:2823-2835 (2003); Ohoka et al., Embo. J. 24:1243-1255 (2005); Ord et al., Biochem. Biophy. Res. Commun. 330:210-218 (2005)). Trib3 was shown to interact with CHOP, a member of the C/EBP family of transcription factors that was not dependent on the lysine in the catalytic core, but dependent on the N-terminal domain (Ohoka et al., supra, 2005).

ATF and C/EBP family members can form dimers and cooperate with each other to activate transcription. Indeed, ATF4 and CHOP cooperate to activate Trib3 promoter activity and functions in a feedback loop control of these proteins during ER stress (Ohoka et al., supra, 2005). No change was reported for Trib1 or Trib2, which suggests functional differences among Trib family members may be determined by their variable N and C termini. Trib1 was shown to inhibit the activity of MEKK-1 mediated activation of the AP-1 promoter and deletion mutants of Trib1 showed that overexpression of the kinase-like domain was sufficient to inhibit stress kinase signaling (Kiss-Toth et al., J. Biol. Chem. 279:42703-42708 (2004); Kiss-Toth et al., supra, 2006). This study also demonstrated that the nuclear localization of Trib1 and 3 was abrogated by deletion of the N-terminal domain. In contrast, Trib2 was localized in a distinct extranuclear region. Mammalian data for Trib2 is limited, with one study identifying Trib2 as a candidate autoantigen in autoimmune uveitis from patient eye samples (Zhang et al., Mol. Immunol. 42:1275-1281 (2005)). A functional role for Trib2 has not previously been described.

The available information on tribbles family members suggests a relationship with C/EBP family members, from Drosophila to mammals, as described above. In hematopoiesis, a well-described role for C/EBPα has been documented in granulocytic differentiation, and more recently, stem cell function (Zhang et al., Immunity 21:853-863 (2004)). There is a biphasic pattern of C/EBPα expression in myeloid differentiation, activated during commitment of multipotential cells to the myeloid lineage, upregulated in granulocytic and downregulated in monocytic differentiation (Radomska et al., Mol. Cell Biol. 18:4301-4314 (1998)). However, C/EBPα can be expressed in macrophages (Hu et al., J. Immunol. 160:2334-2342 (1998)) and has an anti-proliferative role in terminal differentiation of granulocytes (Timchenko et al., Genes Dev. 10:804-815 (1996); Wang et al., Mol. Cell 8:817-828 (2001)). In addition C/EBPα and C/EBPβ form homo-and heterodimers that stabilize the protein and it has been shown that when these proteins do not form dimers, they are degraded by the proteosome (Hattori et al., Oncogene 22:1273-1280 (2003)).

C/EBPα is the only C/EBP family member reported to be associated with AML with mutations found throughout the gene with identifiable cluster regions and preferentially belonging to M1, M2, and M4 FAB subtypes (Leroy et al., Leukemia 19:329-334 (2005)). Studies have shown that a significant number of AML patients with mutations found in C/EBPα have a normal karyotype, mutations can be biallelic and generate dominant-negative truncated forms of C/EBPα (van Waalwijk van Doorn-Khosrovani et al., Hematol. 1 4:31-40 (2003); Gombart et al., Blood 99:1332-1340 (2002); Pabst et al., Nat. Genet. 27:263-270 (2001b); Preudhomme et al., Blood 100:2717-2723 (2002); Snaddon et al., Genes Chromosomes Cancer 37:72-78 (2003)). Loss of C/EBPα activity has also been associated with AML, although C/EBPα knockout mice do not develop AML. They lack granulocytes and eosinophils with an accumulation of immature myeloid cells in fetal liver and peripheral blood (Zhang et al., Proc. Natl. Acad. Sci. U.S.A. 94:569-574 (1997)). Deregulation of C/EBPα by oncogenic fusion proteins is a mechanism involved in AML. AML1-ETO fusion protein has been shown to suppress C/EBPα mRNA expression (Pabst et al., Nat. Med. 7:444-451 (2001a); Westendorf et al., Mol. Cell Biol. 18:322-333 (1998)). C/EBPα mRNA translational inhibition by AML1-MDS1-EVI1 (AME) and CBFb/SMMHC mediated by induction of calreticulin has been documented (Helbling et al., Proc. Natl. Acad. Sci. U.S.A. 101:13312-13317 (2004); Helbling et al., Blood 106:1369-1375 (2005)). In CML, translational inhibition of C/EBPα mRNA by BCR-ABL through induction of hnRNP E2 has been reported (Perrotti et al., Nat. Genet. 30:40-58 (2002)). From these studies it is evident that deregulation of C/EBP in AML and CML plays a functional role in the disease, and underlines the importance of C/EBPα protein. In addition to its associations with hematopoiesis and myeloid leukemogenesis, decreased C/EBPα levels are also associated with lung cancer (see, e.g., Halmos et al., Cancer Res., 62:528-534 (2002)), suggesting that perturbations in C/EBPα levels and signaling may be associated with malignancies and other pathologies outside of the hematopoietic system.

However, until the present invention, a more in-depth understanding remained needed of the causes and conditions associated with AML, and with the malignancies associated with C/EBPα. Also a greater understanding of the biochemistry of AML will enable the development of targeted, efficient therapies for this cancer.

### SUMMARY OF THE INVENTION

The present invention provides a method for diagnosing Acute Myeloid Leukemia (AML) in a patient, wherein the method comprises obtaining a myeloid cell from the patient and assessing Trib2 levels in the myeloid cell. The assessed level of Trib2 in the patient's myeloid cell is then compared to Trib2 levels in a matched myeloid cell obtained from a healthy control subject, from which it is determined whether there is a measurable increase of Trib2 indicative of AML in the patient, as compared with the level for the healthy control subject.

It is an object of the invention to provide a method for diagnosing AML in the patient, by the provided method, wherein C/EBPαp30 levels are assessed in the the myeloid cell, and compared with levels in a matched myeloid cell obtained from a healthy control subject, from which it is determined whether there is a measurable increase of C/EBPαp30 indicative of AML in the patient, as compared with the level for the healthy control subject.

It is also an object of the invention to provide a method for diagnosing AML in the patient, by the provided method, wherein C/EBPαp42 levels are assessed in the the myeloid cell, and compared with levels in a matched myeloid cell obtained from a healthy control subject, from which it is determined whether there is a measurable increase of C/EBPαp42 indicative of AML in the patient, as compared with the level for the healthy control subject.

The methods of the invention may be practiced by assessing nucleotide (e.g., mRNA) or polypeptide levels, and the AML is either M2-AML or M4-AML.

It is a further object to provide a method for inducing maturation of a monocyte from a myeloid cell, comprising administering Trib2 (polynucleotide expressing Trib2 polypeptide, or the Trib2 polypeptide directly) to the myeloid cell.

It is an added object to provide a method for treating a patient having AML, by the method comprising administering a Trib2 inhibitor to the patient, wherein the inhibitor is an inhibitor of either Trib2 polypeptide or Trib2 polynucleotide expression. Such an inhibitor of Trib2 polynucleotide expression is selected from among a Trib2 RNA-binding protein, a Trib2 DNA-binding protein, or a Trib2 antisense polynucleotide. The Trip 2 inhibitor may further be selected from either a polypeptide that binds to a Trib2 polypeptide, such as an antibody, antisense or RNAi composition, or a C/EBPαp30-like polypeptide.

It is yet another object of the invention to provide a method of diagnosing a malignancy associated with Trib2 or C/EBPαp30 or C/EBPαp42 in a patient, the method comprises the steps of obtaining a hematopoietic stem cell from the patient, and assessing the level of Trib2, C/EBPαp30 or C/EBPαp42 in the hematopoietic stem cell. The assessed level of Trib2, C/EBPαp30 or C/EBPαp42 in the patient's hematopoietic stem cell is then compared to Trib2 levels in a matched hematopoietic stem cell obtained from a healthy control subject, from which it is determined whether there is a measurable increase of Trib2, C/EBPαp30 or C/EBPαp42 indicative of a malignancy in the patient, as compared with the level for the healthy control subject. Such malignancy may include AML or lung cancer.

Additional objects, advantages and novel features of the invention will be set forth in part in the description, examples and figures which follow, and in part will become apparent to those skilled in the art on examination of the following, or may be learned by practice of the invention.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIGS. 1A-1G illustrate that Trib2 induces the proliferation of immature myeloid cells in vitro. FIG. 1A depicts methylcellulose colony-forming units (CFU) assays with bone marrow (BM) cells transduced with MigR1 and Trib2 from C57BL/6 bone marrow transplanted chimeric mice. ±SEM is number of colonies of triplicate plates for each condition are tabulated. ++ indicates colony growth and growth in liquid culture. FIG. 1B depicts the morphology of MigR1 (top) and Trib2 (bottom) primary colonies, and FIG. 1C depicts morphology of Trib2 secondary colonies, formed in the indicated cytokines. FIG. 1D depicts the comparative results for 5,000 sorted GFP positive, lineage negative BM cells from Trib2 and MigR1 chimeras plated in methylcellulose containing IL-3, IL-6, SCF, GM-CSF. GM=granulocyte/macrophage colony, G=granulocyte colony, M=macrophage. Data is shown as the percentage of total colonies±SD. FIG. 1E is a photomicrograph of typical Trib2 and MigR1 methylcellulose colonies from FIG. 1D, showing larger GM colonies in Trib2 plates (black arrow). Open arrow=macrophage colony; white arrow=granulocyte colony. FIG. 1F illustrates that single colonies from the primary MigR1 and Trib2 plates in FIG. 1D, replated in liquid culture plus cytokines (IL-3, IL-6, SCF), proliferated continuously (growth=cell expansion, plus media). FIG. 1G is a series of graphs depicting the FACS analysis of MigR1 and Trib2 cells from secondary liquid cultures in FIG. 1F.

FIGS. 2A and 2B show a series of images illustrating the results of bone marrow transduction and transplantation. FIG. 2A is specific for BM. FIG. 2B is specific for spleen, and both illustrate GFP positivity. Left panel: Gr-1^{hi}/CD11b double positive cells (indicative of neutrophils); right panel: F4/80^{+ve}/CD11b^{+ve} double positive cells (indicative of myeloid-derived monocytes) in the GFP^{+ve} and GFP^{-ve} fractions. Results are representative of 3 independent BMT and n=6 mice.

FIGS. 3A-3F illustrate that Trib2-reconstituted mice develop AML. FIG. 3A illustrates the Kaplan-Meier survival curve of mice receiving Trib2 transduced bone marrow compared to MigR1 control. The median survival of Trib2 mice is calculated to be 153 days. FIG. 3B shows images of splenomegaly in Trib2 mice compared to control MigR1 spleen (top spleen), and lymphadenopathy in Trib2 mice. FIG. 3C is a schematic of the Trib2 provirus. FIG. 3D is an image of an electrophoretic gel demonstrating proviral integration in Trib2 mice. DNA preparations were digested with Xba1 (on top) to show the presence of an intact provirus (approx. 4 kb) and BglII (on bottom) that cleaves once in the provirus. Southern blotting performed with an IRES probe. All samples are from primary leukemic mice except lane 1 and 7, which is DNA from a control C57B/6 spleen and MigR1 control spleen respectively, and labeled with the tissue and assigned mouse number from which the DNA was derived. LN=lymph node, Spl=spleen. FIG. 3E depicts Wright-Giemsa staining of peripheral blood, BM and spleen single cell suspensions from MigR1 (left panel) and leukemic Trib2 mice (right panel). Trib2 cells show an immature morphology with myelomonocytic features. Percentage GFP in Trib2 BM and spleen approximates 90-100% and 65-75% respectively. FIG. 3F depicts histopathology of liver sections from Trib2-induced AML. Hematoxylin and Eosin section of spleen as in FIG. 3B showing hypercellularity (low power, top left) with blast morphology (high power, top right) and positive staining for myeloperoxidase (bottom left) and negative staining for Tdt (bottom right).

FIGS. 4A and 4B depict immunophenotypes of primary Trib2-induced AML cells from peripheral blood (PB), lymph nodes (LN), thymus, spleen and bone marrow (BM) compared to control MigR1 mice. FIG. 4A depicts flow cytometric analysis of Gr-1 and CD11b profile and FIG. 4B depicts flow cytometric analysis of c-Kit and F4/80 profile, in the GFP positive fraction of both MigR1 (top panels) and Trib2 (bottom panels). FIG. 4A shows the percentage of cells negative for Gr-1 and CD11b; FIG. 4B shows percentage negative for c-Kit and F4/80. Results are representative of 3 independent BMT and n=7 mice.

FIGS. 5A-5E are a series of images illustrating that Trib2-induced AML is 100% transplantable. FIG. 5A illustrates a Kaplan-meier survival curve of secondary transplants. FIGS. 5B-5E illustrate that immunophenotypes of Trib2-induced AML secondary transplants. Cells from BM, spleen, peripheral blood (PB) and liver were assessed by flow cytometry. FIG. 5B illustrates the Gr-1 and CD11b profile of the GFP population. Percentages indicate cells that are negative for both markers. FIG. 5C illustrates the F4/80 profile of the GFP positive Trib2 cells (black line) shown in a histogram format overlayed with a normal F4/80 profile of C57B/6 control mice (solid line). FIG. 5D illustrates the c-Kit and CD34 profile of the GFP positive population and percentages indicate double positive cells. FIG. 5E illustrates the GFP population is shown on the y axis and the CD16/32 (FcγRII/III) profile on the x axis. Results shown are representative of n=5 mice.

FIGS. 6A-6D are a series of images depicting the real-time RT-PCR analysis of Trib2 expression. FIG. 6A depicts the cDNAs from patients with the indicated AML sub-types and control normal CD34^{+ve} fraction, and cDNA were subjected to real-time RT-PCR with human Trib2-specific primers and probe. Results expressed in percentage, with expression of Trib2 mRNA in AML samples relative to that observed in normal CD34^{+ve} cells, and normalized for 18s rRNA content. Error bars denote the standard deviation of each sample measured in triplicate and in 2 independent experiments. FIG. 6B shows an electrophoretic gel depicting that 32D and U937 cells were transduced with either MigR1 or Trib2. Actin is shown as protein loading control. FIG. 6C shows an electrophoretic gel depicting analysis of C/EBPαp42 and p30 protein expression in primary leukemic samples from BM (93% GFP), spleen (63% GFP), and LN (88% GFP; lymph node) compared to normal levels expressed in CMPs and GMPs from C57B/6 BM (left panel). Levels of C/EBPαp42 and p30 protein expression were compared in total normal C57B/6 BM to that expressed from primary (94% GFP), and secondary (98% GFP), leukemic BM samples (right panel). FIG. 6D shows an electrophoretic gel wherein C/EBPα DNA binding activity was assessed by EMSA using a double-stranded C/EBP binding site from the human G-CSF receptor. Equal amounts of nuclear extracts from U937 cells transduced with MigR1 (lanes 5 and 6), Trib2 (lanes 7 and 8), or C/EBPα (lanes 9 and 10), were sorted for GFP expression. Lanes 3 and 4 are human patient sample 330 (M4-AML) that expressed elevated Trib2, and 12Lanes 1 and 2 are 1 (M5-AML) that had low level expression of Trib2, as shown in FIG. 6A. In lanes 1, 3, 5, 7, 9, 2 μL of C/EBPα antibody was added. ss=supershifted complex; C/EBPα, C/EBPα complex. The same extracts used in lanes 2, 4, 6, 8, and 10 in the top panel were used in an EMSA assay with an OCT-1 probe as a control for integrity and quantity of nuclear binding proteins.

FIGS. 7A-7F depict Trib2 inhibiting the transcriptional activation and functional activity of C/EBPα. FIG. 7A is a schematic of IL-12 promoter containing the C/EBPα binding site. FIG. 7B is a graph depicting that RAW264.7 macrophages were transiently co-transfected with the IL-12 promoter luciferase construct containing the C/EBP WT or mutant consensus sequence, and with empty vector, Trib2 alone, C/EBPα alone, or both Trib2 and C/EBPα. Data presented are mean±SD of triplicate cultures. FIG. 7C shows 32D cells transduced with MigR1, Trib2, or C/EBPα and plated in IL-3 or G-CSF. Percentage CD11b and was assessed at 4 days. Data is presented as a percentage relative to MigR1 control and representative of 3 independent experiments. FIG. 7D illustrates the percentage GFP expression was assessed in 32D cells as in FIG. 7C. Data is presented as a percentage relative to day 0 transduction efficiency of each sample and representative of 3 independent experiments. FIG. 7E is an electrophoretic gel of U937 (top panel) and 32D (lower panel) cells transduced and sorted for MigR1 or Trib2. FIG. 7F is an electrophoretic gel depicting that 293T cells were transfected with empty vector (lane 1), myc-tagged Trib2 (lane 2), HA-tagged C/EBPα (lane 3), or co-transfected with both (lanes 4 and 5), and treated with 10 μM MG132 for 2 hours (lane 4). Trib2 was immunoprecipitated using a Myc antibody and western blotting performed with HA and Myc antibodies on immunoprecipitates (top panel) and total lysates (lower panel).

FIGS. 8A-8E show the results from C57BL/6 mice lethally irradiated and reconstituted with BM cells transduced with MigR1 and Trib2. FIG. 8A depicts a flow cytometric analysis of GFP^{+ve}/CD11b^{+ve} myeloid-derived dendritic cell (DC) population from BM (left panel) and spleen (right panel) of MigR1 (top) and Trib2 (bottom) chimeric mice as defined by MHC II^{+ve}/CD11c^{+ve} (percentages shown). FIG. 8B depicts a flow cytometric analysis of GFP^{+ve}/CD11b^{+ve} myeloid-derived macrophage cell population from BM (left panel) and spleen (right panel) of MigR1 (top) and Trib2 (bottom) chimeric mice as defined by MHC II^{+ve}/F4/80^{+ve} (percentages shown). Results are representative of 3 independent BMT at 9-14 weeks post-transplant. FIG. 8C is a flow cytometric analysis of in vitro derived DC defined by CD11c^{+ve}/CD11b^{+ve} (left panel) showing the activation status defined by CD86 and MHC II expression (right panel). FIG. 8D is a flow cytometric analysis of in vitro derived macrophage defined by F4/80+^{ve}/CD11b^{+ve} (left panel); activation status defined by CD86 and MHC II expression (right panel). FIG. 8E shows the total cell numbers of in vitro derived macrophage and DC cultures. Data presented is the mean±SEM of triplicate cultures.

FIGS. 9A-9E comprise a series of graphs depicting that RAW264.7 macrophages were transiently co-transfected with the NFκB consensus promoter luciferase construct and with empty vector, Trib2 alone, C/EBPα alone or both Trib2 and C/EBPα. Luciferase activity was measured following LPS (100 ng/ml) treatment for 8 hours, 24 hours post-transfection. Reporter luciferase activity for each sample was normalized to the Renilla luciferase activity for the same sample. Data presented are mean±SD of triplicate cultures. FIGS. 9B-9E illustrate that in vitro derived macrophage and DC cultures at day 8 were stimulated with LPS for 1 day. ELISA was performed to detect IL-12 (FIGS. 10B and 10D) and IL-6 (FIGS. 9C and 9E) production. Data presented is the mean±SD of triplicate cultures.

### DETAILED DESCRIPTION OF THE INVENTION

The present invention relates to the diagnosis and treatment of AML, and in particular, the diagnosis and the treatment of AML by way of Trib2. For the first time Trib2 has been shown to be an oncogenic protein involved in the pathogenesis of AML. Trib2 chimeric mice develop AML with a phenotype similar to human, specifically to human M2/M4 AML, with elevated white blood cells counts with blast-like and immature myeloid morphology. Provided, therefore, are also methods for the diagnosis and treatment of M2-AML and M4-AML, as well as AML in general. Because the disclosed elevated expression of Trib2 mRNA correlates with human M2/M4 AML in a patient screen, the present invention, therefore, also relates to the diagnosis and treatment of AML by Trib2 mRNA.

Furthermore, Trib2 plays a role in monocyte/macrophage development, as well as in the inhibition of granulocytic differentiation and C/EBPα function. Therefore, the present invention also relates to the stimulation of monocyte/macrophage development by way of Trib2. Accordingly, the invention also relates to the inhibition of granulocyte development by way of Trib2.

Methods of Diagnosing AML

The present invention provides a method of diagnosing AML in a patient. As described in greater detail below, it has now been shown that the level of Trib2 in a myeloid cell of a patient can be used to assess the presence or absence of AML in a patient. Further, the present invention features a method of diagnosing other malignancies and diseases in the patient. This is in part because C/EBPα downregulation has been associated with additional malignancies. One of skill in the art will understand how to identify a malignancy or disorder associated with C/EBPα downregulation.

In one embodiment, an elevated level of Trib2 in a myeloid cell of a patient is an indication that the patient is afflicted with AML. In another embodiment, a method of diagnosing a patient as being afflicted with AML includes obtaining at least one myeloid cell from the patient, assessing the level of Trib2 in the cell, and comparing the assessed level of Trib2 to the level of Trib2 in an otherwise identical myeloid cell obtained from a healthy control subject not afflicted with AML. One of skill in the art will understand how to conduct additional or further testing to confirm or further define the status of such a patient, and therefore, such methods need not be discussed in detail herein.

The invention should not be construed to be limited to a myeloid cell, however. Rather, the invention properly includes any hematopoietic stem cell. The invention also includes any cell in the hematopoietic stem cell lineage. That is, the invention also includes a cell at any developmental stage between a hematopoietic stem cell and a myeloid cell. This is because it has been shown that AML can begin in a hematopoietic stem cell (HSC), and as set forth in greater detail below, the present invention is useful to identify, characterize, and treat AML, among other things. As will be understood by the skilled artisan, when armed with the disclosure set forth herein, a HSC can also be a leukemic stem cell. By way of a non-limiting example, the present invention also has applicability to the mobilization of an HSC to the peripheral blood by a chemical agent, or by a biological agent, such as a lymphokine, including, but not limited to, granulocyte macrophage colony stimulating factor (GM-CSF).

An “otherwise identical hematopoietic stem cell” is a hematopoietic stem cell that is obtained from a similar source, and is of a similar genetic and phenotypic lineage. Further, an “otherwise identical myeloid cell” is a myeloid cell that is obtained from a similar source, and is of a similar genetic and phenotypic lineage. In a non-limiting example, a hematopoietic stem cell can be derived from the bone marrow of a patient. Other sources of hematopoietic stem cells include, but are not limited to, multi-potent progenitor cells and common myeloid progenitors. Other sources of hematopoietic stems cells will be understood by the skilled artisan and are known in the art.

By way of a non-limiting example, a myeloid cell can be derived from the bone marrow of a patient. Other sources of myeloid cells will be understood by the skilled artisan. Furthermore, a non-limiting example of an otherwise identical myeloid cell includes, but is not limited to, a myeloid cell that is differentiating towards a macrophage lineage and a myeloid cell that is differentiating towards a granulocyte lineage. This applies equally to a myeloid cell obtained from a patient afflicted with, or belieived to be afflicted with AML, as well as a myeloid cell obtained from a healthy control subject.

The assessment of Trib2 in a patient can be by any mechanism either now known in the art or yet to be discovered. Any such method is within the scope and field of the present invention. Methods of assessing Trib2 levels in a myeloid cell include, but are not limited to, assessing Trib2 RNA, assessing Trib2 DNA, assessing Trib2 protein, and assessing an identifiable fragment of each.

The present invention also features a method of diagnosing AML in a patient by assessing C/EBPαp30, and the level of C/EBPαp30 in a myeloid cell of the patient can be used to assess the presence or absence of AML in the patient. In one aspect of the invention, an elevated level of C/EBPαp30 in a myeloid cell of a patient is an indication that the patient is afflicted with AML. In an embodiment, a method of diagnosing a patient as being afflicted with AML includes obtaining at least one myeloid cell from the patient, assessing the level of C/EBPαp30 in the cell, and comparing the assessed level of C/EBPαp30 to the level of C/EBPαp30 in an otherwise identical myeloid cell obtained from a healthy control subject not afflicted with AML.

In another embodiment of the invention, an elevated level of C/EBPαp30, in conjunction with a decreased level of C/EBPαp42 in a myeloid cell of a patient is an indication that the patient is afflicted with AML. The level of C/EBPαp42 is assessed as described for C/EBPαp30.

Another embodiment of the present invention is particularly useful for diagnosing FAB subtypes M2 or M4 AML in a patient. However, the invention should not be construed to be limited to diagnosing any particular type of AML. By way of a non-limiting example, the present invention is useful for diagnosing AML in general, and more specifically, for diagnosing M2-AML, M4-AML, as well as other types of AML. Furthermore, the present invention is useful for diagnosing lung cancer, as well as other tumors.

Methods of Treating AML in a Patient

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

To “treat” a disease as the term is used herein, means to reduce the frequency of the disease or disorder reducing the frequency with which a symptom of the one or more symptoms disease or disorder is experienced by an animal. A “measurable increase” refers to a greater increase in the level or quantity of a substance. By way of a non-limiting example, an increase in the level of Trib2 mRNA in a first sample, as compared to the level of Trib2 mRNA in a second sample, is any level of increase that is measurable using any method known in the art or other method yet to be developed, provided that the two measured amounts are not equivalent. Similarly, a “measurable decrease” as used herein refers to a lessened level or quantity of a substance.

The “treatment of a patient,” as used herein, refers to the reduction of the symptoms and/or causes of a disease, condition or disorder in a patient.

As used herein the term “subject” is used interchangeably with the term “patient.” A “healthy control subject” refers to a subject that is not afflicted with a disease or disorder to which the subject is being compared. By way of a non-limiting example, for comparison with a subject having AML, the corresponding “healthy control subject” is a subject that does not have AML. The term “patient,” as used herein, refers to a mammal that is either afflicted with a disease or disorder, or a mammal that is healthy and non-afflicted with that disease or disorder. By way of a non-limiting example, the severity of AML can be reduced according to methods of the present invention, in which Trib2 polypeptide is inhibited. Treatment encompasses the partial inhibition of Trib2, as well as the complete inhibition of Trib2 in a myeloid cell. Treatment also encompasses the partial alleviation of AML symptoms, as well as the complete alleviation of AML symptoms in a patient afflicted with AML.

The present invention provides a method of treating a patient having AML, wherein an increased level of Trib2 in a subject correlates to the disease state of AML. That is, a measurably increased amount of Trib2 polypeptide or polynucleotide (e.g., Trib2 mRNA) in a myeloid cell of a patient, when compared with the level of Trib2 polypeptide or polynucleotide from a healthy control subject, is an indication that the patient is afflicted with AML. Therefore, the inhibition of Trib2 polypeptide or polynucleotide in such a patient can be used as a treatment for AML, to reverse the condition or conditions created by overexpression of Trib2 or by elevated levels of Trib2.

A “polynucleotide” means a single strand or parallel and anti-parallel strands of a nucleic acid. Thus, a polynucleotide may be either a single-stranded or a double-stranded nucleic acid. A portion of a polynucleotide means at least about twenty sequential nucleotide residues of the polynucleotide. It is understood that a portion of a polynucleotide may include every nucleotide residue of the polynucleotide. The term “oligonucleotide or oligomer”, as used herein, refers to a molecule comprised of two or more deoxyribonucleotides or ribonucleotides, preferably more than three. Its exact size will depend on many factors, which in turn depend on the ultimate function or use of the oligonucleotide. An oligonucleotide may be derived synthetically or by cloning. Conventional notation is used herein to describe polynucleotide sequences: the left-hand end of a single-stranded polynucleotide sequence is the 5′-end; the left-hand direction of a double-stranded polynucleotide sequence is referred to as the 5′-direction, and the invention further includes recombinant polynucleotides.

A “recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well. A recombinant polypeptide is one which is produced by expression of a recombinant polynucleotide.

As used herein, a “therapeutic” treatment refers to one administered to a patient who exhibits signs of pathology for the purpose of diminishing or eliminating those signs, and/or decreasing or diminishing the frequency, duration and intensity of the signs. Thus, a “therapeutic protein,” or “therapeutic compound” refers to protein or compound that improves or maintains the health of the cell expressing the protein or that of a cell in proximity to the cell expressing the protein. Numerous exemplary therapeutic proteins and compounds are widely-known in the art and are not listed here since they are well-known to the artisan. An “effective amount” of such a protein or compound, therefore, is that amount of compound which is sufficient to provide a detectable effect to a cell to which the compound is administered when compared to an otherwise identical cell to which the compound is not administered.

In one embodiment, a method according to the present invention of treating a patient afflicted with AML includes administering to a patient an inhibitor of Trib2. As will be understood by the skilled artisan when armed with the disclosure set forth herein, inhibitor of Trib2 include, but are not limited to, a Trib2 RNA-binding compound, a Trib2 RNAi, a Trib2 DNA-binding compound, and a Trib2 polypeptide-binding compound. Such compounds include a small molecule, a naturally-occurring product, a polypeptide, a polynucleotide, a lipid, a carbohydrate, or any combination thereof.

In one aspect, a Trib2 inhibitor is an antisense molecule, the identification, preparation and use of which are described in greater detail elsewhere. In another aspect, a Trib2 inhibitor is a polypeptide that can bind to Trib2 polypeptide, dimerize with Trib2 polypeptide, or otherwise effectively prevent Trib2 polypeptide from participating in the typical Trib2 roles within a myeloid cell. In yet another aspect, a Trib2 inhibitor is a small molecule, such as a serine/threonine kinase inhibitor, which has affinity for Trib2, and therefore, can effectively prevent Trib2 polypeptide from participating in the typical Trib2 roles within a myeloid cell. While, in still another aspect, a Trib2 inhibitor is an antibody or antibody fragment, wherein the antibody or antibody fragment has a particular affinity for Trib2 polypeptide, and therefore, can effectively prevent Trib2 polypeptide from participating in the typical Trib2 roles within a myeloid cell. Methods of identifying, producing and using such antibodies or antibody fragments are either known or described in greater detail elsewhere herein.

As shown, Trib2 plays a role in the development and progression of AML. Trib2 also plays a role in the preferential maturation of a myeloid cell into a monocyte/macrophage lineage, rather than maturation to granulocyte lineage. Therefore, inhibitors of Trib2 polypeptides or polynucleotides can modulate AML or myeloid cell maturation, by way of Trib2.

Methods of Affecting Myeloid Cell Growth and Development

The present invention also provides a method of inducing maturation of a blood cell from a hematopoietic stem cell progenitor. As more fully set forth elsewhere herein, elevated levels of Trib2 can be used to affect hematopoietic stem cell maturation due to the role of the Trib2 in AML, and more particularly, the hematopoietic stem cell-maturing effect of increased levels of Trib2. In one aspect, the present invention includes a method of inducing maturation of a blood cell from a myeloid progenitor. As more fully set forth elsewhere herein, elevated levels of Trib2 can be used to affect myeloid cell maturation due to the role of the Trib2 in AML, and more particularly the myeloid cell-maturing effect of increased levels of Trib2.

In yet another embodiment of the invention, a method of inducing development of a blood cell from a myeloid cell comprises administering Trib2 to a myeloid cell. Upon administration, Trib2 induces the maturation of the myeloid cell towards the monocyte lineage. Therefore, the present invention is useful for the production of a monocyte. In yet another embodiment, a method of inducing development of a blood cell comprises administering Trib2 to a hematopoietic stem cell.

The present invention also features the production of a macrophage. In an embodiment of the invention, a method of inducing development of a blood cell from a myeloid cell comprises administering Trib2 to a myeloid cell. Upon administration, Trib2 induces the maturation of the myeloid cell towards the macrophage lineage.

Accordingly, when armed with the present disclosure, the skilled artisan will understand the utility of monocytes, macrophages, and the production thereof. By way of a non-limiting example, the production of a macrophage according to the present invention is also useful for the production of activated macrophages and giant cells. Similarly, the production of a macrophage according to the present invention is useful for the production, via differentiation of a macrophage, of osteoclasts and microglia, as well as dendritic cells.

As described in detail elsewhere herein, Trib2 can be administered to a myeloid cell in various forms. In one embodiment, Trib2 polypeptide is administered to a cell. In another embodiment, Trib2 polynucleotide is administered to a cell. Trib2 polynucleotide (e.g., cDNA) can be administered in a nucleic acid vector, and Trib2 polypeptide expressed therefrom within the myeloid cell. Trib2 RNA can also be administered to a cell, and Trib2 polypeptide expressed therefrom using the endogenous cellular machinery. Thus, the method of the present invention is not limited to any particular manner in which Trib2 is provided to a cell or to a mammal; rather, the invention encompasses various methods whereby Trib2, and/or a portion thereof, is administered to a cell or to a mammal.

Trib2 polynucleotide or polypeptide can be administered to a mammal via a variety of routes. Further, the dosage and amounts administered depend on numerous factors which are discussed more fully elsewhere herein. Pharmaceutical compositions and other relevant methods for administering polynucleotides and polypeptides are known in the art and are described, for instance, in Genaro, ed. (Remington's Pharmaceutical Sciences, Mack Publishing Co., Easton, Pa. (1985)), which is incorporated herein by reference. The amount of Trib2 administered, whether it is administered as polypeptide or as polynucleotide, is sufficient to detectably modulate the symptoms of AML or to modulate the maturation of a myeloid cell towards a monocyte/macrophage lineage.

When Trib2 is administered by administering a nucleic acid encoding the protein, the nucleic acid can be administered “naked” (e.g., substantially free of any other substance with which a nucleic acid is typically associated such as protein, and the like). Alternatively, the nucleic acid can be encapsulated or otherwise associated with another substance capable of facilitating the introduction of the nucleic acid into a cell. Such nucleic acid delivery techniques are described elsewhere herein and are well-known in the art and are described in, for example, Sambrook et al., supra, and Ausubel et al., supra.

An amount of Trib2 polynucleotide or polypeptide sufficient to detectably modulate the symptoms of AML or to modulate the maturation of a myeloid cell towards a monocyte/macrophage lineage can be readily determined using any of the assays disclosed herein as well as methods well-known in the art. “Modulate” refers to the alteration of a process or activity from one state or condition to another. For example, modulation of Trib2 activity refers to the inhibition of Trib2 activity. Modulation of Trib2 activity also refers to the increase of Trib2 activity. For example, Trib2 activity may be modulated by increasing Trib2 protein activity through one or more amino acid mutations. Modulators are discussed in greater detail below.

Patients that can benefit from administration of a Trib2 polynucleotide or polypeptide will be understood based on the disclosure set forth herein. In one aspect, a patient that can benefit from the administration of a Trib2 polynucleotide or polypeptide is a patient afflicted with AML, or a patient that is in an early stage of development of AML, prior to full development of AML. In another aspect, a patient that can benefit from administration of a Trib2 polynucleotide or polypeptide is a patient in need of an increased level of monocytes or macrophages.

Nucleic Acids

The invention includes an isolated nucleic acid encoding Trib2, for the purpose of administration of such a nucleic acid to a patient in need thereof, according to the methods of the present invention. While the invention is exemplified with the isolated human Trib2 nucleotide sequence (SEQID No:1), it will be understood that mutants, fragments, variants and homologs of Trib2 can be used according to the methods of the invention, if such mutants, fragments, variants and homologs of Trib2 have the activity of Trib2 as set forth herein. That is, nucleic acids encoding polypeptides other than wild type human Trib2 are encompassed by the present invention, provided that such molecules have the ability to induce maturation of myeloid cells towards a monocyte/macrophage lineage.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. By example, the mRNA sequence for a Trib2 homolog is presented as it appears in Drosophila (SEQID No:3).

The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence. A,C, G, T and U are used as understood in the art to abbreviate the commonly occurring nucleic acid bases.

An isolated nucleic acid is “substantially pure” meaning that the nucleic acid, or also as used in reference to the encoded protein, is a nucleic acid or protein preparation that is generally lacking in other cellular components with which it is normally associated in vivo. That is, as used herein, the term “substantially pure” describes a compound, e.g., a nucleic acid, protein or polypeptide, which has been separated from components which naturally accompany it. Typically, a compound is substantially pure when at least about 10%, preferably at least about 20%, more preferably at least about 50%, still more preferably at least about 75%, even more preferably at least about 90%, and most preferably at least about 99% of the total material (by volume, by wet or dry weight, or by mole percent or mole fraction) in a sample is the compound of interest. Purity can be measured by any appropriate method, e.g., by column chromatography, gel electrophoresis or. HPLC analysis.

A compound, e.g., a nucleic acid, a protein or polypeptide is, therefore, “substantially purified” when it is essentially free of naturally associated components or when it is separated from the native contaminants which accompany it in its natural state. Thus, a substantially pure nucleic acid composition refers to a nucleic acid sequence which has been purified from the sequences which flank it in a naturally occurring state, e.g., a DNA fragment which has been removed from the sequences which are normally adjacent to the fragment in a genome in which it naturally occurs.

“Gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the human proteins described herein are within the scope of the invention. Nucleic acid molecules corresponding to natural allelic variants and homologs of a cDNA of the invention can be isolated based on their identity to human nucleic acid molecules using the human cDNAs, or a portion thereof, as a hybridization probe according to standard hybridization techniques under stringent hybridization conditions.

“Encoding” or “encoded” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Thus, the use of the term “DNA encoding” should be construed to include the DNA sequence which encodes the desired protein and any necessary 5′ or 3′ untranslated regions accompanying the actual coding sequence.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of an mRNA molecule also consists of the nucleotide residues of the mRNA molecule which are matched with an anticodon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. A first region of an oligonucleotide “flanks” a second region of the oligonucleotide if the two regions are adjacent to one another or if the two regions are separated by no more than about 1000 nucleotide residues, and preferably no more than about 100 nucleotide residues.

A “DNA segment” or “fragment” refers to a molecule comprising a linear stretch of nucleotides wherein the nucleotides are present in a sequence that encodes, through the genetic code, a molecule comprising a linear sequence of amino acid residues that is referred to as a protein, a protein fragment, or a polypeptide. “Gene” refers to a single polypeptide chain or protein, and as used herein includes the 5′ and 3′ ends. The polypeptide can be encoded by a full-length sequence or any portion of the coding sequence, so long as the functional activity of the protein is retained. A “complementary DNA” or “cDNA” includes recombinant genes synthesized by reverse transcription of messenger RNA (“mRNA”) lacking intervening sequences (introns).

“Homologous” as used herein, refers to the subunit sequence similarity between two polymeric molecules, e.g., between two nucleic acid molecules, e.g., two DNA molecules or two RNA molecules, or between two polypeptide molecules (homologs). When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. As used herein, “homology” is used synonymously with “identity.” Moreover, when the term is used herein to refer to the nucleic acids and proteins, it should be construed to be applied to homology at both the nucleic acid and the amino acid levels.

The determination of percent identity between two nucleotide or amino acid sequences can be accomplished using a mathematical algorithm. For example, a mathematical algorithm useful for comparing two sequences is the algorithm of Karlin and Altschul (Proc. Natl. Acad. Sci. USA 87:2264-2268 (1990)), modified as in Proc. Natl. Acad. Sci. USA 90:5873-5877 (1993)). This algorithm is incorporated into the NBLAST and XBLAST programs of Altschul et al., J. Mol. Biol. 215:403-410 (1990)), and can be accessed, for example, at the National Center for Biotechnology Information (NCBI) world wide web site of the National Library of Medicine (NLM) at the National Institutes of Health (NIH) using the BLAST program. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul et al., Nucleic Acids Res. 25:3389-3402 (1997)). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used. These programs are publicly available at, e.g., the website for the National Center for Biotechnology Information (NCBI) world wide web site of the National Library of Medicine at the National Institutes of Health.

The percent identity between two sequences can be determined using techniques similar to those described above, with or without allowing gaps. In calculating percent identity, typically exact matches are counted.

A “variant” or “allelic or species variant” of a protein or nucleic acid is meant to refer to a molecule substantially similar in structure and biological activity to either the protein or nucleic acid. Thus, provided that two molecules possess a common activity and may substitute for each other, they are considered variants as that term is used herein even if the composition or secondary, tertiary, or quaternary structure of one of the molecules is not identical to that found in the other, or if the amino acid or nucleotide sequence is not identical.

Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. As used herein, “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

By the term “exogenous nucleic acid” is meant that the nucleic acid has been introduced into a cell or an animal using technology which has been developed for the purpose of facilitating the introduction of a nucleic acid into a cell or an animal. The term “expression of a nucleic acid” means the synthesis of the protein product encoded by the nucleic acid. More specifically, expression is the process by which a structural gene produces a polypeptide. It involves transcription of the gene into mRNA, and the translation of such mRNA into a polypeptide.

A cell that comprises an exogenous nucleic acid is referred to as a “recombinant cell.” Such a cell may be a eukaryotic cell or a prokaryotic cell. A gene expressed in a recombinant cell, wherein the gene comprises a recombinant polynucleotide, produces a “recombinant polypeptide.”

The invention includes a nucleic acid encoding Trib2, wherein a nucleic acid encoding a tag polypeptide is covalently linked thereto. That is, the invention encompasses a chimeric nucleic acid wherein the nucleic acid sequences encoding a “tag” polypeptide is covalently liked to the nucleic acid encoding the Trib2 polypeptide (SEQID No:2). A “tag” polypeptide refers to any protein which, when linked by a peptide bond to a protein of interest, may be used to localize the protein, to purify it from a cell extract, to immobilize it for use in binding assays, or to otherwise study its biological properties and/or function. Such tag polypeptides are well known in the art and include, for instance, green fluorescent protein (GFP), myc, myc-pyruvate kinase (myc-PK), hexahistidine, maltose biding protein (MBP), an influenza virus hemagglutinin tag polypeptide, a flag tag polypeptide (FLAG), and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

A chimeric (i.e., fusion) protein containing a tag epitope can be immobilized on a resin which binds the tag. Such tag epitopes and resins which specifically bind them are well known in the art and include, for example, tag epitopes comprising a plurality of sequential histidine residues (His6), which allows isolation of a chimeric protein comprising such an epitope on nickel-nitrilotriacetic acid-agarose, a hemagglutinin (HA) tag epitope allowing a chimeric protein comprising such an epitope to bind with an anti-HA-monoclonal antibody affinity matrix, a myc tag epitope allowing a chimeric protein comprising such an epitope to bind with an anti-myc-monoclonal antibody affinity matrix, a glutathione-S-transferase tag epitope, and a maltose binding protein (MBP) tag epitope, which can induce binding between a protein comprising such an epitope and a glutathione- or maltose-Sepharose column, respectively. Production of proteins comprising such tag epitopes is well known in the art and is described in standard treatises, such as Sambrook et al., Molecular Cloning, A Laboratory Approach, Cold Spring Harbor Press, Cold Spring Harbor, N.Y. (2001), and Ausubel et al., Current Protocols in Molecular Biology, John Wiley & Sons, NY (2002). Likewise, antibodies to the tag epitope (e.g., anti-HA, anti-myc antibody 9E10, and the like) allow detection and localization of the fusion protein in, for example, Western blots, ELISA assays, and immunostaining of cells.

In other related aspects, the invention includes a vector which comprises an isolated nucleic acid encoding Trib2. Preferably, the vector is capable of directing expression of Trib2 in a vector-containing cell. Vectors suitable for use in the present invention include, but are not limited to, vectors which facilitate the generation of multiple copies of nucleic acid encoding Trib2 or which facilitate expression of Trib2 protein in either prokaryotic or eukaryotic cells or both. Thus, the invention should not be construed to be limited to any known vector system, but rather should include all suitable known or heretofore unknown vectors which facilitate the generation of multiple copies of Trib2 encoding nucleic acid, or which facilitate the expression of Trib2 in a cell. Examples of suitable vectors include bacteriophage T7-based expression vectors for replication and expression in bacteria, the pMSXND expression vector for replication and expression in mammalian cells and baculovirus-derived vectors for replication and expression in insect cells. Adenoviruses, retrovirus and other viral vectors are also contemplated in the invention.

The invention further includes an isolated nucleic acid having a sequence which is in the antisense orientation (i.e., is complementary) to all or a portion of the isolated nucleic acid encoding Trib2. “Antisense nucleic acid sequence,” “antisense sequence,” “antisense DNA molecule” and “antisense gene” all refer to pseudogenes which are constructed by reversing the orientation of the gene with regard to its promoter, so that the antisense strand is transcribed. The term also refers to the antisense strand of RNA or of cDNA which compliments the strand of DNA encoding the protein or peptide of interest. In either case, when introduced into a cell under the control of a promoter, the anti-sense nucleic acid sequence inhibits the synthesis of the protein of interest from the endogenous gene. The inhibition appears to depend on the formation of an RNA-RNA or cDNA-RNA duplex in the nucleus or in the cytoplasm. Thus, if the antisense gene is stably introduced into a cultured cell, the normal processing and/or transport is affected if a sense-antisense duplex forms in the nucleus; or if antisense RNA is introduced into the cytoplasm of the cell, the expression or translation of the endogenous product is inhibited.”

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

Antisense nucleic acid sequences can further include modifications which can affect the biological activity of the antisense molecule, or its manner or rate of expression. Such modifications can also include, e.g., mutations, insertions, deletions, or substitutions of one or more nucleotides that do not affect the function of the antisense molecule, but which may affect intracellular localization. Modifications include, but are not limited to, 5-fluorouracil, 5-bromouracil, 5-chlorouracil, 5-iodouracil, hypoxanthine, xanthine, 4-acetylcytosine, 5-(carboxyhydroxymethyl uracil, 5-carboxyhydroxymethyl-2-thiouridine, 5-carboxymethylaminomethyl uracil, dihydrouracil, beta-D-galactosylqueosine, inosine, N6-isopentyladenine, 1-methylguanine, 1-methylinosine, 2,2 dimethylguanine, 2-methyladenine, 2-methylguanine, 3-methylcytosine, 5-methylcytosine, N6-adenine, 7-methylguanine, 5-methylaminomethyluracil, 5-methylaminomethyl-2-thioracil, beta-D-mannosylqueosine, 5′-methoxycarboxymethyluracil, 5-methyluracil, 2-methylthio-N6-isopentenyladenine, uracil-5 oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methy-2-thiouracil, 3-(3-amino-3-N-2-carboxypropyl)uracil, and 2,6-diaminopurine.

The antisense nucleic acid sequence can determine an uninterrupted antisense RNA sequence or it can include one or more introns. The terms “complementary” and “antisense” as used herein, are not entirely synonymous. “Antisense” refers particularly to the nucleic acid sequence of the non-coding strand of a double stranded DNA molecule encoding a protein, or to a sequence which is substantially homologous to the non-coding strand. “Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

In one aspect, the invention includes an antisense RNA sequence characterized in that it can bind to mRNA encoding Trib2 and thereby inhibit synthesis of Trib2. As above, vectors, including those in which the nucleic acid is operatively linked to promoter/regulatory elements, and cells comprising an antisense Trib2 isolated nucleic acid sequence are contemplated in the invention.

Polypeptides

The invention additionally includes an isolated polypeptide encoded by a Trib2 nucleic acid. Polypeptide refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer. Conventional notation is also used herein to portray polypeptide sequences: the left-hand end of a polypeptide sequence is the amino-terminus; the right-hand end of a polypeptide sequence is the carboxyl-terminus.

As described above, an isolated or substantially pure protein or polypeptide composition refers to a protein or polypeptide which has been purified from components with which it is normally associated in its naturally occurring state. A substantially pure peptide can be purified by following known procedures for protein purification, wherein an immunological, enzymatic or other assay is used to monitor purification at each stage in the procedure. Protein purification methods are well known in the art, and are described, for example in Deutscher et al. (1990, In: Guide to Protein Purification, Harcourt Brace Jovanovich, San Diego).

As stated above, the invention should in no way be construed to be limited to wild type human Trib2 polypeptide (SEQID No:2). Rather, the invention should be construed to include any isolated Trib2 polypeptide or any mutant, variant, or homolog thereof, having the biological activity of Trib2 as defined elsewhere herein. For example, conservative amino acid changes may be made, which although they alter the primary sequence of the protein or peptide, do not normally alter its function, as recognized in the art. By example, the polypeptide sequence for a Trib2 homolog is presented as it appears in mouse (SEQID No:4).

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Antibodies

The invention further provides an antibody that specifically binds with Trib2, or a fragment thereof In a preferred embodiment, the invention includes an antibody that inhibits the biological activity of Trib2. The antibody is useful for the identification for Trib2 in a diagnostic assay for the determination of the levels of Trib2 in a mammal having a disease associated with Trib2 levels. In addition, an antibody that specifically binds Trib2 is useful for blocking the interaction between Trib2 and a Trib2-binding component found in a myeloid cell, and is therefore useful in a therapeutic setting for treatment of Trib2 related disease (e.g., AML), as described herein.

The generation of antibodies that specifically bind to Trib2 is described briefly herein. By the term “specifically bind to,” or “specific binding” as used herein, is meant a compound, e.g., a protein, a nucleic acid, an antibody, and the like, which recognizes and binds a specific molecule, but does not substantially recognize or bind other molecules in a sample. However, the invention should be construed to include any and all antibodies which can be made that specifically bind to Trib2. For example, the generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al., In: Antibodies, A Laboratory Manual, Cold Spring Harbor, N.Y. (1998) and in Tuszynski et al., Blood 72:109-115 (1988)). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al., Critical Rev. in Immunol. 12(3,4):125-168 (1992) and the references cited therein. Further, the antibody of the invention may be “humanized” using the technology described in Wright et al., supra and in the references cited therein, and in Gu et al., Thrombosis and Hematocyst 77(4):755-759 (1997).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage, which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., supra.

Processes such as those described above, have been developed for the production of human antibodies using M13 bacteriophage display (Burton et al., Adv. Immunol. 57:191-280 (1994)). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. See, e.g., Marks et al., J. Mol. Biol. 222:581-597 (1991). Panning of such phage for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, Nature Medicine 1:837-839 (1995); de Kruif et al., J. Mol. Biol. 248:97-105 (1995)).

Modulators of Trib2 and Trib2 Activity

The invention provides molecules which are capable of modulating the expression and/or activity of Trib2 in a cell or in a bodily fluid of a mammal. By the term “modulator” of Trib2 activity, as used herein, is meant a compound that affects the biological activity of Trib2, as defined herein, wherein the activity is either higher or lower in the presence of the modulator compared with the activity of Trib2 in the absence of the modulator.

Thus, a modulator can be an inhibitor or an enhancer of Trib2 expression or activity. Modulators that inhibit Trib2 expression include, but are not limited to, antisense molecules and ribozymes which bind to and/or cleave Trib2 encoding nucleic acid. The invention also provides for inhibitors of Trib2 which serve to reduce or eliminate Trib2 protein molecules. Such inhibitors can be antisense nucleic acids or ribozymes, as described above. Inhibitors can also be double stranded RNA molecules that serve to reduce the level of Trib2 mRNA by RNA interference as described (Elbashir et al., Nature 411:428-429 (2001); Carthew, Curr. Opin. Cell Biol. 13:244-248 (2001)).

It is a relatively simple matter, once armed with the present disclosure, to identify a modulator of Trib2 expression or of its biological activity. For example, cells which naturally express Trib2, or which express Trib2 following transfection with Trib2 encoding nucleic acid may be contacted with a test compound. The level of expression of Trib2 in the presence or absence of the test compound is then measured, wherein a higher or lower level of expression of Trib2 in the presence of the test compound compared with the level of Trib2 expression in the absence of the test compound, is an indication that the test compound is a modulator of Trib2 expression. When the level of Trib2 is elevated in the presence of the test compound compared with the level of expression of Trib2 in the absence of the test compound, the test compound is considered to be an enhancer of Trib2 expression. Conversely, when the level of Trib2 expression is reduced in the presence of the test compound compared with the level of expression of Trib2 in the absence of the test compound, the test compound is considered to be an inhibitor of Trib2 expression.

Similarly, Trib2 biological activity can be measured, for example, in myeloid cells. In this instance, the level of the biological activity of Trib2 produced by cells in the presence or absence of a test compound is measured, wherein a higher or lower level of activity of Trib2 in the presence of the test compound compared with the level of Trib2 activity in the absence of the test compound, is an indication that the test compound is a modulator of Trib2 biological activity. When the level of Trib2 activity is elevated in the presence of the test compound compared with the level of activity of Trib2 in the absence of the test compound, the test compound is considered to be an enhancer of Trib2 biological activity. Conversely, when the level of Trib2 activity is reduced in the presence of the test compound compared with the level of activity of Trib2 in the absence of the test compound, the test compound is considered to be an inhibitor of Trib2 biological activity.

Expression of Trib2 may be measured using any ordinary molecular biology technology, such as using RT-PCR technology, RNAse protection, Northern blotting and the like. Alternatively, affects on expression may be measured by operably linking the Trib2 promoter sequence to a suitable reporter gene and transfecting cells with the resulting DNA construct. Promoter activity responsive to the test compound may be measured by measuring the level of the reporter gene expression in cells contacted with the test compound and comparing the level of reporter gene expression in those cells with cells not contacted with the test compound. Suitable reporter genes include, but are not limited to beta-galactosidase, chloramphenicol acetyl transferase, green fluorescent protein, and the like.

Preferred reagents for detection of Trib2 nucleic acid include, but are not limited to, a nucleic acid complementary to the nucleic acid encoding Trib2. Preferred reagents for detection of Trib2 protein include, but are not limited to, an antibody. It is further preferred that these reagents be labeled to facilitate detection of Trib2 nucleic acid or protein. One skilled in the art would appreciate, based on the disclosure herein, that regents for detection of Trib2 can be labeled using a variety of suitable labels including a radioisotope, a bioluminescent compound, a chemiluminescent compound, a fluorescent compound, a metal chelate, or an enzyme.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Examples

The following materials and methods were used in the Examples as described.

Luciferase Reporter Assay: RAW264.7 macrohages were transfected with either pcDNA control expression vector, Trib2 (390 ng), C/EBPα (10 ng) and IL-12 p40 (100 ng) wild-type and mutant promoter-luciferase (firefly) constructs. 24 hours post transfection, cells were treated with 100 ng/ml LPS for 8 hours and reporter luciferase activity was measured using the Dual-Luciferase reporter assay kit (Promega Corp., Madison, Wis.), per manufacturers instructions. Co-transfection of the Renilla-luciferase expression vector pRL-TK (10 ng) (Promega) was used as an internal control. The data was normalized for transfection efficiency by dividing firefly luciferase activity by that of the Renilla luciferase.

Constructs and retroviruses: A 1032 bp fragment encoding for the entire murine Trib2 cDNA was subcloned into the pcDNA3.1/myc-HIS plasmid, and MigR1 vector. C/EBPα rat cDNA was subcloned from MigR1 vector (previously described, (Keeshan et al., Blood 102:1267-1275 (2003)) into the pcDNA3.1/myc-HIS plasmid. The IL-12 p40 promoter containing the genomic fragment −700 to +54 of the IL-12 p40 gene was amplified by PCR from C57BL6 genomic DNA and cloned into the pGL3-basic vector (Promega) and site directed mutagenensis of the C/EBP binding site (−93 to −89) was performed using the QuickChange kit (Stratagene, La Jolla, Calif.) according to manufacturer's instructions.

Bone marrow transduction and transplantation: C57BL/6 mice (B6) were obtained from Taconic Laboratories. Experiments were performed according to guidelines from the National Institutes of Health and with an approved protocol from the University of Pennsylvania Animal Care and Use Committee. Transduction of B6 bone marrow cells with retroviral supernatant produced through transient transfection of 293T cells and transfer of these cells into lethally irradiated recipients were performed as described previously (Pui et al., Immunity 11:299-308 (1999)). Briefly, bone marrow (BM) cells were collected from 6- to 10-week-old mice 4 days after intravenous administration of 5-FU (5 mg), retrovirally transduced ex vivo in the presence of IL-3, IL-6 and SCF and 0.2-1×10⁶ cells were injected intravenously into lethally irradiated (900 rads) B6 recipients. Chimeric mice were maintained on antibiotics for 2 weeks and analyzed at least 6 weeks after transplantation. Secondary transplants were performed by injecting 2×10⁶ nucleated BM or spleen cells from the primary leukemic mice into sublethally irradiated (600 rads) B6 mice.

Tissues were fixed in formalin and sectioned and stained with hematoxylin and eosin, MPO and TDT for histological analysis. Blood smears and cytospin preparations were stained with Hema3 staining kit.

Immunoprecipitation and Western Blotting: 293T cells in 10 cm dishes were transfected with 3 μg pcDNA3.1/myc-HIS-Trib2 and 2 μ\tilde{g} MigR1-C/EBPα. After 36 hours, co-transfected cells were treated with 10 μM MG132 for 1 hour and NEM (N-Ethylmaleimide) (CalbiochemNovaBiochem, Corp., La Jolla, Calif.) for 30 seconds, washed once with 1× PBS containing NEM (10 mM), then protein lysates taken using modified RIPA buffer (Tris-HCL-50 mM, NP40-0.5%, Na-deoxycholate-0.25%, NaCl-150 mM, EDTA-1 mM, PMSF-1 mM, Na₃Vo₄-1 mM, NaF-1 mM, NEM-20 mM, supplemented with a cocktail of serine and cysteine protease inhibitors (Complete EDTA-free; Hoffmann-La Roche Ltd). Supernatants were precleared with 50 μl 1:1 slurry of protein A agarose beads for 1 hour. 3 mg of precleared lysates were incubated overnight with 20 μl 1:1 slurry protein A beads coated with 5 μg MYC antibody. The beads were washed 3 times in lysis buffer and resuspended in 2× SDS loading buffer. For detection of C/EBPα in leukemic samples, cells were lysed directly in 2× SDS buffer. Western blotting was performed according to standard procedures. Antibodies; anti-C/EBPα (Sc-61, Santa Cruz Biotechnology, Inc, Santa Cruz, Calif.), anti-HA (HA.11, Covance, Princeton, N.J.), anti-MYC (myc1-9E10).

Methylcellulose Clonogenic Assays: Bone marrow cells were isolated from MigR1 and Trib2 transplanted mice and either sorted for GFP alone or for GFP and negative for lineage marker expression (CD3, CD4, CD8, B220, Gr-1, Ter119, CD19, MHC II, IL-7Rα). Cells were plated in triplicate in methylcellulose media (Methocult™ M3231, StemCell Technologies, Inc., Vancouver, BC) supplemented with cytokines (M-CSF-10 ng/ml, G-CSF-10 ng/ml, GM-CSF-10 ng/ml, IL-3-10 ng/ml, IL-6-10 ng/ml, SCF-50 ng/ml (PeproTech Inc., Princeton, N.J., and BD Pharmingen, San Diego, Calif.). Colonies were scored after 9 days in primary plates and morphologically assessed by modified Wright-Giemsa staining (HEMA 3 stain kit) of cytospin preparations. 15,000 cells from primary plates were transferred to secondary and tertiary methylcellulose plates containing the indicated cytokines and counted (10 days secondary, 8 days tertiary), then transferred to liquid culture containing RPMI, 10% FBS and cytokines. Single colonies were transferred from primary plates to RPMI media containing IL-3, IL-6, SCF, in 24 well plates and assessed for growth after 11 days. FACS analysis for Lineage markers (Ter119, CD3, CD8, Gr-1, CD4, B220, CD19, CD11c), c-Kit, Sca-1, CD11b, F4/80 and CD16/32 was performed.

Electrophoretic Mobility Shift Assay (EMSA): EMSA assays were performed as previously described (Keeshan et al., supra, 2003) except for the following modifications. Nuclear extracts were obtained using Nuclear Extract Kit (Active Motif) as per manufacturers instructions. The G-CSF receptor promoter oligonucleotide (C/EBP site underlined) had the sequence 5′ AAGGTGTTGCAATCCCCAGC 3′ (SEQID NO:5). The Oct-1 consensus oligonucleotide was obtained from Santa Cruz Biotechnology. 2 μl of C/EBPα (sc-61x, Santa Cruz Biotechnology) was used for supershift experiments.

Quantitative RT-PCR: RNA was isolated using the RNEasy kit (Qiagen, Chatsworth, Calif.), digested with Dnase 1 and used for reverse transcription according to manufacturers instructions (Superscript II™ kit, Invitrogen, Carlsbad, Calif.). Validated human Trib2 (SEQID No:1) and 18s rRNA primer/probe sets and TaqMan® Universal PCR Master Mix (Applied Biosystems, Foster City, Calif.) were used for qRT-PCR and analyzed on the ABI Prism 7900 sequence detection system (Applied Biosystems).

DNA analysis: Southern blotting was performed according to standard procedures. Briefly, high molecular weight DNA was isolated from snap-frozen tissues, digested with appropriate restriction enzymes, and southern blotting performed using QuikHyb® (Stratagene) buffer and labeled IRES probe.

Flow Cytometry. Cell suspensions were stained in PBS/2% FBS after blocking with Rat/Mouse IgG (Sigma Chemical Company, St. Louis, Mo.). Cells were sorted on a MoFlo (Cytomation; Dako, Carpinteria, Calif.) cell sorter. Analytical flow cytometry was performed on FACS Calibur (Becton Dickinson, Lincoln Park, N.J.) and analyzed using FlowJo software (Tree Star, Inc., Ashland, Oreg.). The following antibodies were used: Phycoerythrin (PE)-anti-CD11b (Mac-1, M1/70), PE-anti-Gr-1 (RB6-8C5), PE-anti-Sca1 (Ly-6A/E), PE-anti-CD16/32 (FcγII/III), PE-anti-B220 (RA3-6B2), PE-anti-CD19 (1D3), PE-anti-CD4 (L3T4), PE-anti-CD8α (Ly.2), PE-anti-CD3 (145-2C11), PE-anti-Ter-119 (Ly-76), PE-anti-IA^{b} (MHC II/AF6-120-1), PE-anti-CD86 (B7.2), biotin-anti-CD34 (RAM34), allophycocyanin (APC)-anti-c-Kit (2B8/CD117), APC-anti-CD11c (HL3). Caltag: biotin-anti-F4/80, APC-anti-CD11b, eBiosciences: PE-anti-IL7R α (CD127/A7R34), APC-anti-CD16/32 (Pharmingen). Biotinylated antibodies were revealed with Streptavidin-PerCP (Pharmingen).

Cell Culture: For bone marrow-derived macrophage and dendritic cell cultures (BM-φ and BM-DC), GFP-sorted bone marrow cells were cultured in DMEM, 10% FBS, 30% L-Sup (supernatant from L-929 cells, as a source of GM-CSF) for BM-φ and RPMI, 10% FBS, 20 ng/ml GM-CSF and 5 ng/ml IL-4 for BM-DC. BM-φ media was replaced every 2 days, BM-DC media was added to existing plates every 2 days. 8 day cultures were harvested, cell number assessed, FACS analysis performed and 50,000 cells/well in triplicate plated in 96 well plates and stimulated with 100 ng/ml LPS for 1 day. ELISA performed to test for IL-6 and IL-12. 32D cells were maintained in IMDM, 10% FBS, 10% WEHI-conditioned media. For differentiation assays, 32D cells were plated in 5 ng/ml IL-3 or 25 ng/ml G-CSF and assessed for granulocytic differentiation by FACS analysis and morphological criteria. U937 cells were maintained in RPMI, 10% FBS, 10 mM HEPES. RAW264.7 macrophage cell line was maintained in DMEM and 10% FBS.

### Example 1

### Trib2 Induces the Proliferation of Immature Myeloid Cells in vitro

Trib2 was identified in a microarray screen of T-ALL cell lines performed to identify potential novel genes involved in leukemogensis. To test the role for Trib2 during hematopoiesis, lethally irradiated C57B/6 mice were reconstituted with transduced progenitors as previously described (Pui et al., Immunity 11:299-308 (1999)). Equal titre retroviruses were used to give similar transduction efficiencies, and engraftment was assessed at 4-6 weeks post transplant using GFP as a marker. FACS analysis of peripheral blood revealed similar engraftment efficiencies for MigR1 and Trib2 (data not shown).

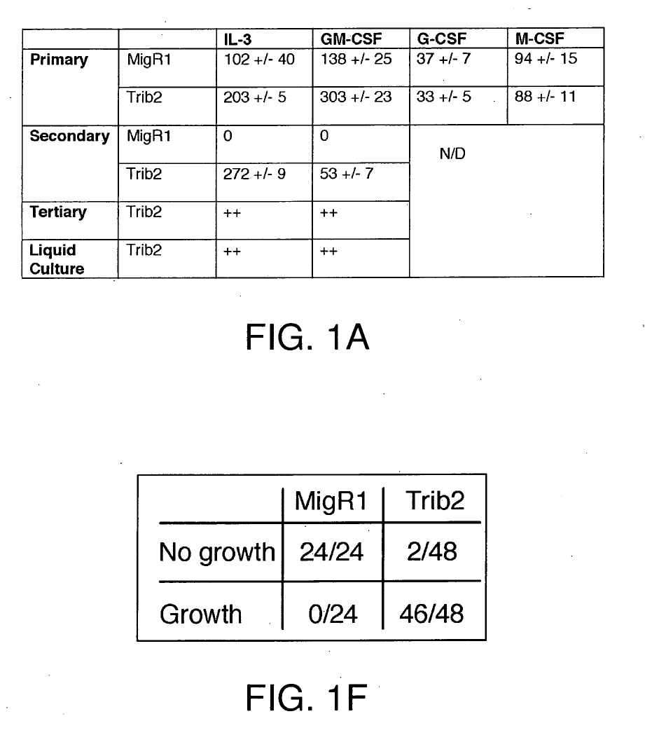

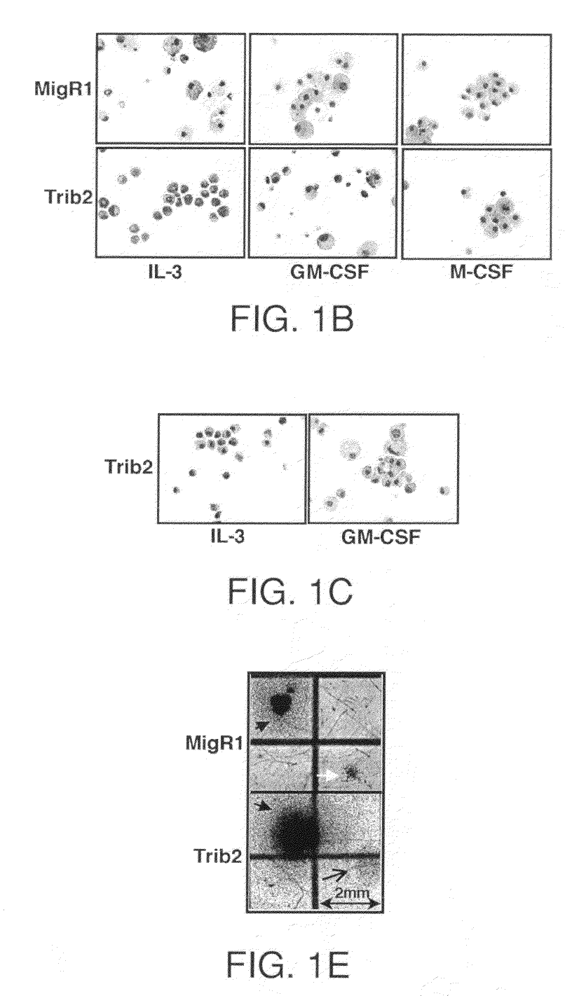

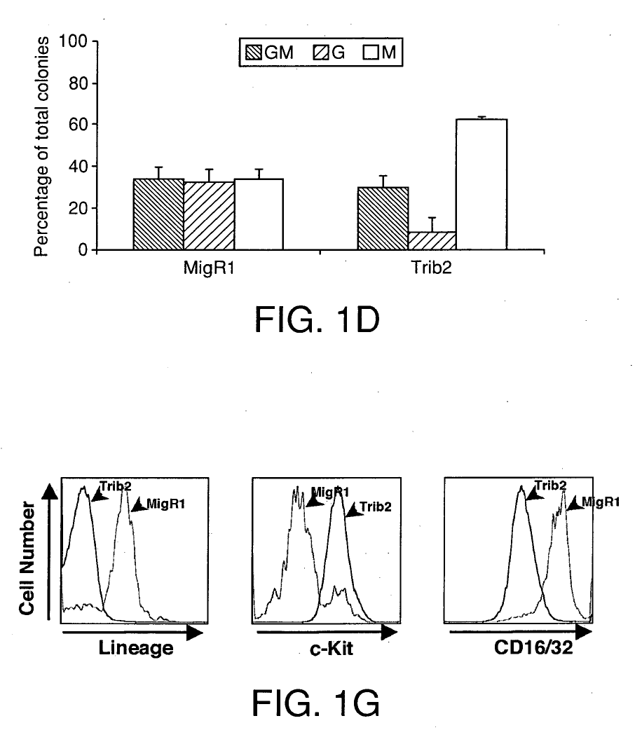

To test the myeloid potential and repopulating ability of Trib2 transduced cells, GFP positive cells were sorted from bone marrow of MigR1 and Trib2 chimeras at 9-10 weeks post-transplant, and equal numbers of MigR1 and Trib2 cells were plated in methylcellulose in different cytokine conditions, i.e., IL-3, GM-CSF, G-CSF, or M-CSF, and colony forming units (CFU's) were assessed. 25,000 GFP positive cells sorted from BM of 9-10 week MigR1 and Trib2 chimeric mice were plated in methylcellulose in the indicated cytokines. Colonies with >50 cells were scored after primary plating (9 days), secondary replating in IL-3 and GM-CSF (15,000 cells, 10 days), tertiary replating (8 days, 15,000 cells), and liquid culture (8 days). Colony number and size were similar between MigR1 and Trib2 in G-CSF and M-CSF. However, significant differences in colony number and size were observed in IL-3 (Trib2; 203±5 versus MigR1; 102±40) and GM-CSF (Trib2; 303±23 versus MigR1; 138±25), conditions that promote differentiation and allow for progenitor proliferation (FIG. 1A).

The larger colonies produced in Trib2 plates indicate more primitive progenitors with a higher proliferative potential. Thus, 10 day primary IL-3 and GM-CSF plates were washed and 15,000 cells were replated in new methylcellulose plates to test the ability to form colonies upon secondary plating. Cells from MigR1 primary colonies were unable to form colonies in IL-3 or GM-CSF upon secondary replating, whereas Trib2 cells were capable of secondary colony formation in IL-3 (272±9) and GM-CSF (53±7), and could successfully be transferred from secondary plates after 10 days to form tertiary colonies after 8 days and could successfully proliferate in liquid culture for a further 8 days (upon which cells were stored in liquid nitrogen) (FIG. 1a).

In a morphological assessment of primary colonies for 5,000 sorted GFP positive, lineage negative BM cells from Trib2 and MigR1 chimeras plated in triplicate in methylcellulose containing IL-3, IL-6, SCF, GM-CSF, it was seen in comparative results that in the IL-3 and GM-CSF plates, Trib2 colonies contained cells with blast-like features i.e. round nucleus, higher nuclear/cytoplasmic ratio in primary plates (FIG. 1B), and these cells were evident in secondary plates (FIG. 1C).

To determine the clonogenicity of Trib2-transduced cells, bone marrow cells from MigR1 and Trib2 chimeras were lineage depleted and sorted for GFP expression. 5000 cells were plated in methylcellulose with cytokines that promote progenitor proliferation and differentiation (IL-3, IL-6, SCF, and GM-CSF). After 9 days, larger colonies morphologically similar to granulocyte/macrophage (GM) colonies, were again evident in 24-well Trib2 plates (FIG. 1E), while macrophage (M) colonies were increased, and granulocytic (G) colonies were reduced (FIG. 1D). Single colonies were randomly picked and replated in liquid media plus IL-3, IL-6 and SCF in 24 well plates and assessed for secondary proliferation. MigR1 colonies were unable to proliferate upon secondary replating, whereas 46 out of 48 Trib2 colonies were shown to proliferate continuously (FIG. 1F) over 11 days. Trib2 cells from secondary plates were lineage negative and positive for c-Kit and CD16/32 expression, as assessed by flow cytometry (FIG. 1G), and phenotypically similar to myeloid progenitor cells, whereas MigR1 cells from secondary plating had differentiated as determined by lineage marker expression. Trib2 cells stained negative/low for Scal, and are negative for CD11b and F4/80 staining.

### Example 2

### Trib2 Promotes Monocytic and Inhibits Granulocytic Differentiation in vivo

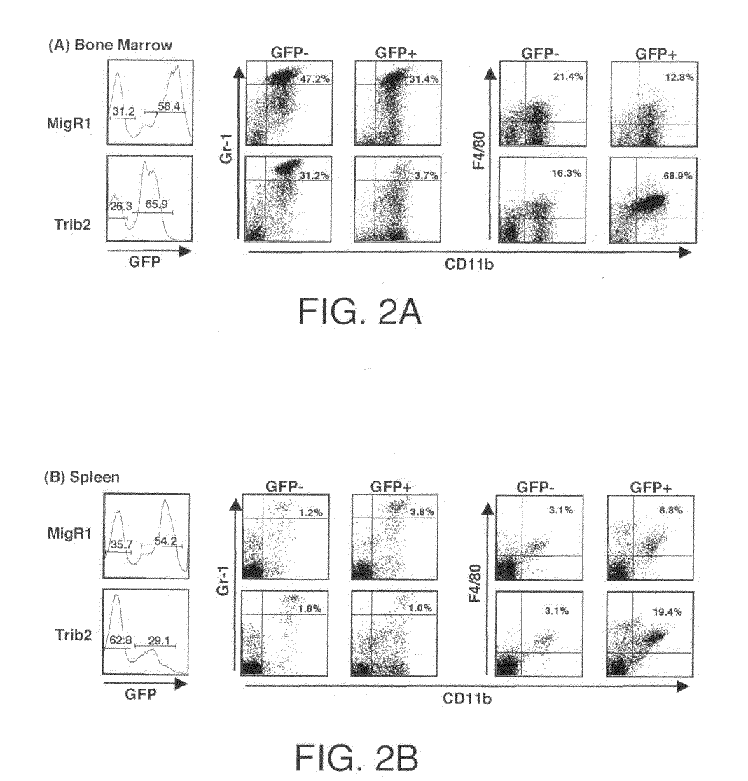

To assess the in vivo characteristics of Trib2 expressing cells in hematopoiesis, flow cytometric analysis was performed from bone marrow and spleen cells from MigR1 and Trib2 chimeras at 9-14 weeks post transplant. C57BL/6 mice were lethally irradiated and reconstituted with BM cells transduced with MigR1 and Trib2, resulting in a GFP percentage that was similar in MigR1 and Trib2 mice (FIG. 2). The percentage of granulocytes in vivo (CD11b^{+ve}/Gr-1^{hi}) was reduced in the GFP positive population of Trib2 mice in the bone marrow at this time point (FIG. 2A). Importantly, the percentage of monocytes in vivo (CD11b^{+ve}/F4/80^{+ve}) was significantly increased in the GFP positive population of both the bone marrow and spleen of Trib2 mice (FIG. 2B). These data support the results from in vitro methylcellulose assays shown in FIG. 1, and implicate a role for Trib2 in myeloid lineage decisions. The thymus and lymph nodes of Trib2 chimeras at this time point contained GFP positive cells and exhibited normal lineage distribution (data not shown).

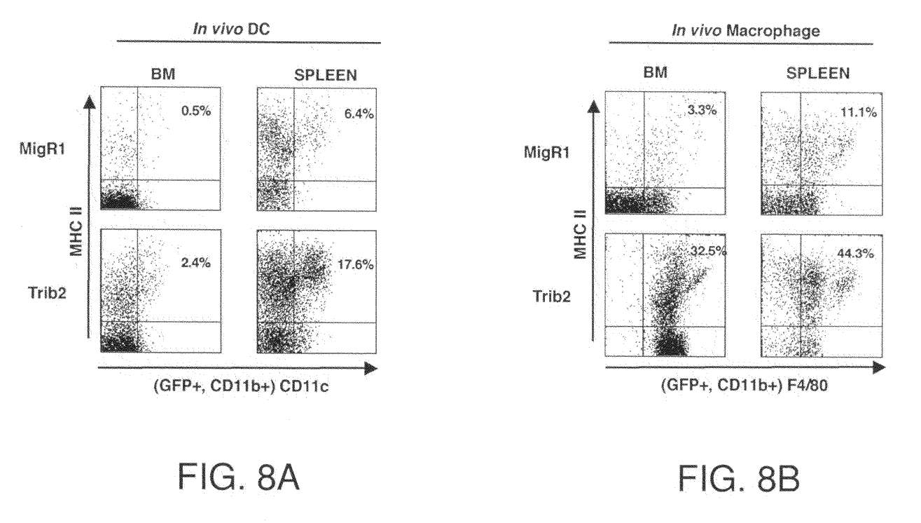

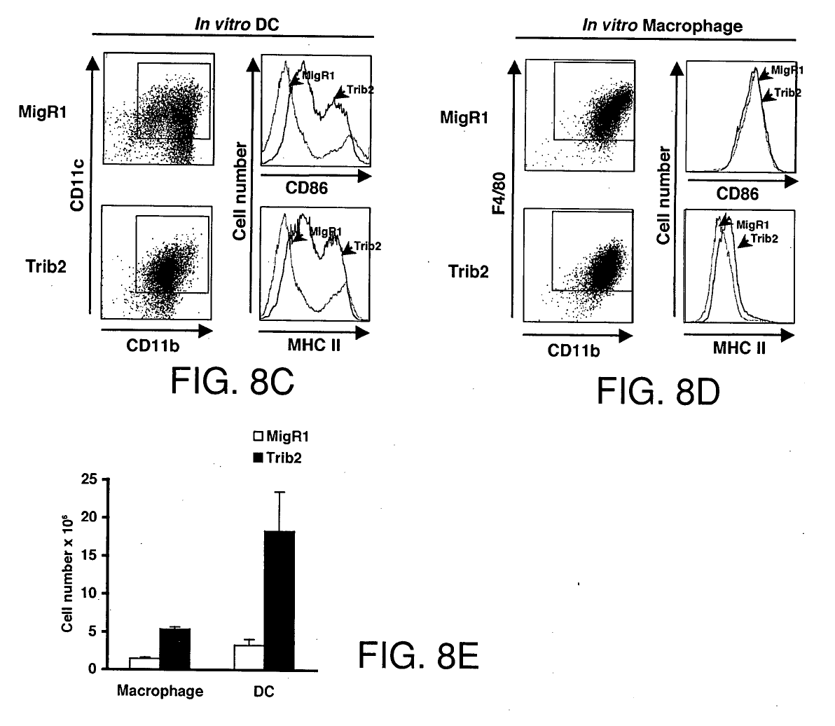

Flow cytometric analysis was performed on 9-14 week MigR1 and Trib2 chimeric mice to detect in vivo myeloid macrophages and dendritic cells (DC) to determine if Trib2 alters these cell types. The bone marrow (BM) and spleen of the Trib2 chimeric mice displayed elevated levels of CD11b^{+ve}, CD11c^{+ve}/MHC II^{+ve} DCs in the GFP positive population compared to MigR1 control mice (FIG. 8A). A more pronounced elevation of CD11b^{+ve}, F4/80^{+ve}/MHC II^{+ve} macrophages were detected in the GFP positive population of Trib2 mice (FIG. 8B). Furthermore, the in vitro production of bone marrow derived macrophages (BM-macrophage) and dendritic cells (BM-DC) was significantly more efficient with bone marrow from Trib2 mice compared to MigR1 control mice (FIG. 8E). Flow cytometric analysis of activation markers in macrophage and DC cultures revealed that the BM (in vitro derived DC) sorted from GM-CSF and Trib2 chimeric mice and cultured in GM-CSF and IL-4 (in vitro derived DC) or L-sup (in vitro derived macrophage) for 8 days, exhibited increased activation as assessed by CD86 and MHC II staining, and BM-macrophages displayed slight differences compared to MigR1 controls (FIGS. 8C and 8D). These in vitro BM-DC and macrophages were shown to be functional in a phagocytosis assay.

### Example 3

### Trib2 Reconstituted Mice Develop AML

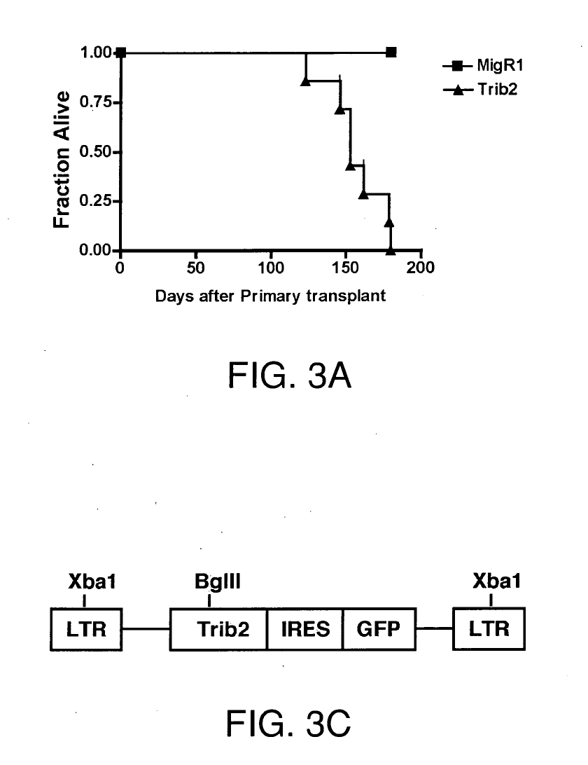

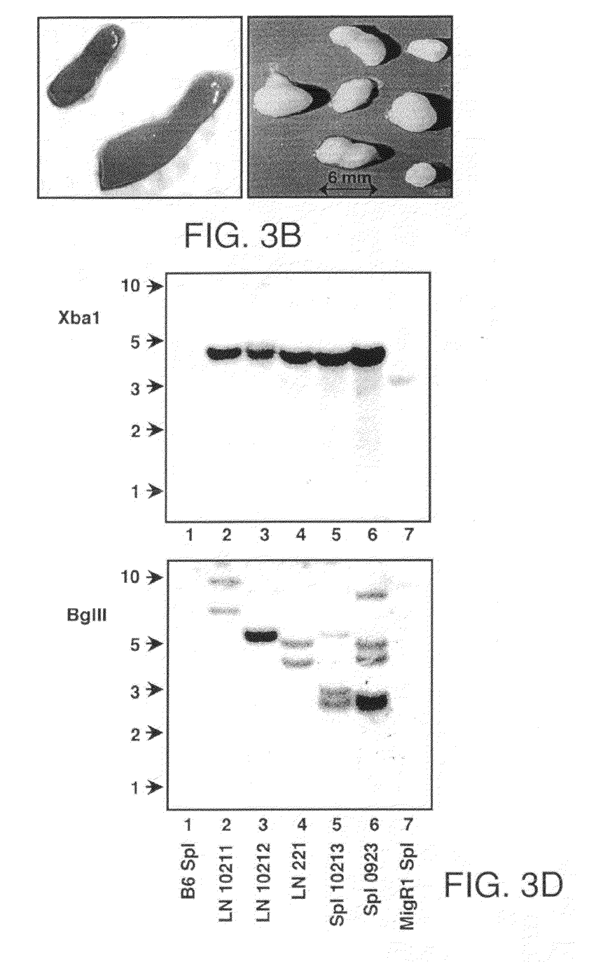

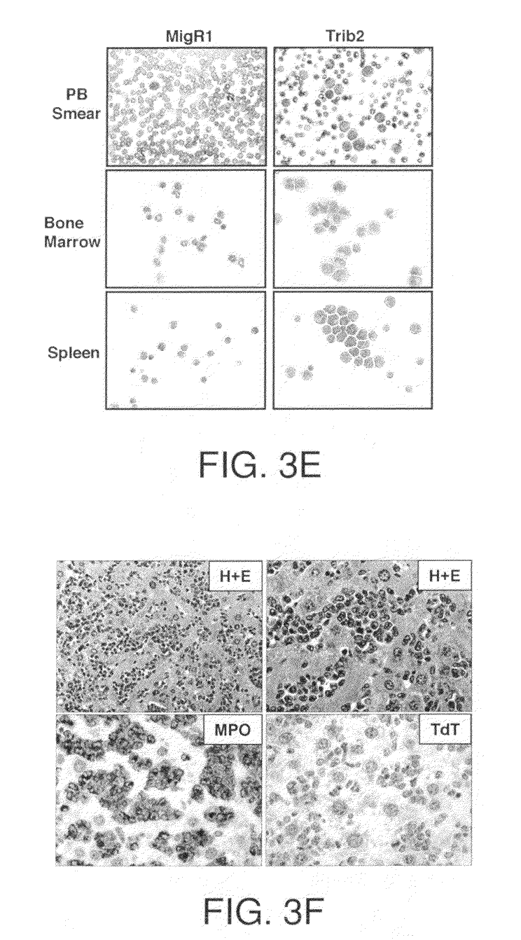

Mice reconstituted with Trib2 and MigR1 were observed, and it was found that 100% of Trib2 chimeras died with a median survival of 153 days (FIG. 3A; Table 1). White blood cells (WBC) counts were monitored and found to be significantly elevated prior to death. All animals displayed splenomegaly and lymphadenopathy as shown in FIG. 3B; Table 1. Spleens were 2 to 6 times bigger than control spleens, and WBC counts were elevated to 150×10⁶/ml in some mice (Table 1). The GFP percentage in Trib2 mice was typically 90-100% in bone marrow, 65-75% in spleen and 80-95% in lymph nodes.

To demonstrate that the leukemic cells contained an intact provirus, DNA was taken from lymph nodes and spleen, digested with Xba1, which cleaves once in the 5′ and 3′ LTR's, and probed with IRES sequences contained in the provirus (FIG. 3C). All tumors contained the expected 4 kb provirus, and control MigR1 spleen DNA expectantly contained the 2.9 kb provirus (FIG. 3d, top panel). To enumerate the proviruses, DNA was digested with BglII and probed with IRES sequences. All tumors were found to be either monoclonal (FIG. 3D, lower panel, lanes 2-4), or oligoclonal (lane 5 and 6), as determined by the intensity of the bands. This finding suggests that the disease tissue arose from a single cell that had sustained double (lanes 2 and 4) or multiple (lanes 3, 5 and 6) retroviral infections.

To determine if the elevated WBC counts were due to circulating myeloblasts and had characteristics of AML, peripheral blood smears and cytospin preparations were performed. WBC counts were clearly elevated in the blood smears with morphological features of blasts cells and immature myelomonocytic cells, i.e., round nucleus, some kidney shaped, high nuclear/cytoplasmic ratio, reduced red blood cells (FIG. 3E). Furthermore, the bone marrow and spleens were clearly packed with these leukemic cells with a notable absence of normal granulocytes (FIG. 3E).

Tissues were fixed in formalin and sectioned and stained for histological analysis. Hematoxylin and eosin staining of liver sections showed hypercellularity at low magnification, and at higher magnification these cells are clearly myeloblasts with some differentiation (FIG. 3F, top panel). The cells stained positive for myeloperoxidase and negative for TdT, characteristic of AML (FIG. 3F, lower panels). These histological analyses revealed that the Trib2-induced leukemias resemble human M2 AML.

TABLE 1
Summary of Trib2 primary bone marrow transplants.

| Primary | Days post | | Spleen | | Secondary |
|---|---|---|---|---|---|
| Trib2 BMT | transplant | WBC × 10⁶ | WT (gr) | Leukemia | Transplant |
| 1 | 123 | 148 | 0.35 | AML | N/D |
| 2 | 146 | 75 | 0.45 | AML | N/D |
| 3 | 180 | 150 | 0.66 | AML | Yes |
| 4 | 153 | 129 | 0.63 | AML | Yes |
| 5 | 153 | 52 | 0.41 | AML | Yes |
| 6 | 162 | 122 | 0.50 | AML | N/D |
| 7 | 179 | 28 | 0.21 | AML | N/D |
| MigR1 | 146 | 7 | 0.09 | NO | |

Results summarize 3 independent experiments. Days post transplant refers to time of death or to onset of terminal symptoms (cachexia, decreased activity, and increased WBC counts determined by tail bleeding.) A representative MigR1 control mouse is shown as comparison.
N/D = not done.

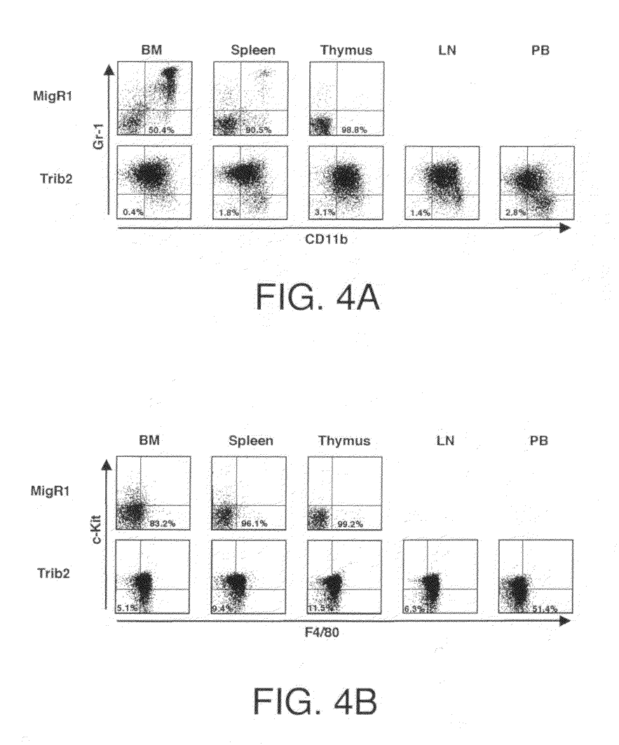

To further characterize the leukemic cells from Trib2-induced AML mice, cells were assessed by flow cytometry for marker expression. Compared to MigR1 chimeras, Trib2 mice contained few to no cells that were negative for CD11b or Gr-1 (0.4%-3.1%). The CD11b/Gr-1 profile was not typical of normal granulocytes/monocytes, as cells displayed intermediate levels of both markers similar to the staining profile characteristic of myeloid leukemic cells. This CD11b/Gr-1 profile was evident throughout the mice, as leukemic cells infiltrated the bone marrow, spleen, thymus, lymph node and peripheral blood (FIG. 4A). Also, Trib2 leukemic cells stained positive for c-Kit and F4/80 in infiltrated organs yet this was not as pronounced in the peripheral blood (FIG. 4B). None of the Trib2 leukemic cells were recognized by antibodies reacting to T or B lymphocytes. These cell surface markers on the Trib2 leukemic cells reinforce the blast-like myelomonocytic characteristics of the AML induced by Trib2.

### Example 4

### Trib2-Induced AML is 100% Transplantable

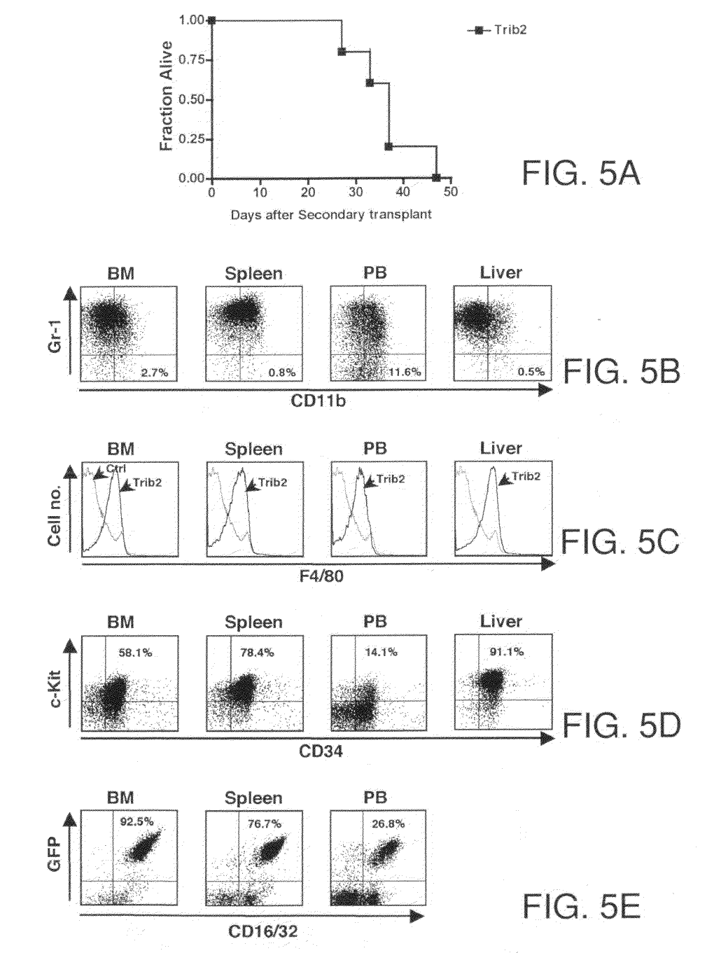

To further establish the malignancy of the Trib2 disease, the transplantability of primary leukemia to secondary hosts was performed. 2×10⁶ primary leukemic cells from the bone marrow and spleens of primary leukemic mice were transplanted into sublethally irradiated (600 rads) secondary recipients and monitored for signs of disease, i.e., cachexia and decreased activity. 100% of secondary recipients developed AML with an average latency of 36 days (FIG. 5A; Table 2). Significant increase in spleen weight was observed in all mice with splenic nodules present in 20% mice. WBC counts were approximately double the normal counts obtained in MigR1 mice. Infiltration of leukemic cells was evident in the bone marrow, spleen and liver (Table 2). Liver enlargement was present in all secondary recipients.

Immunophenotypic analysis of the secondary disease demonstrated characteristics similar to the primary disease shown in FIG. 4. Percentage GFP reached >90% in the bone marrow while the peripheral blood remained <30% in secondary transplants and GFP positive cells were mostly CD16/32 (FcγRII/III) positive indicative of myeloid lineage (FIG. 5E). The Gr-1/CD11b profile was similar to primary leukemic cells, with percentages of double negative cells remaining low (0.5-11.6%), however a reduction in CD11b expression was apparent (FIG. 5B). F4/80 expression remains elevated (FIG. 5C). In addition to cells expressing c-Kit, secondary leukemic cells also express CD34 (FIG. 5D). This c-Kit/CD34 profile, which is uniform in the liver, resembles that of CMPs and GMPs.

These data demonstrate the transplantability of Trib2-induced primary AML. In addition, cell lines have been derived from primary AML samples from bone marrow and peripheral blood and continue to proliferate in a growth-dependent manner.

TABLE 2
Summary of Trib2 secondary transplants.

| Secondary | Days post | | | |
|---|---|---|---|---|
| Trib2 BMT | transplant | WBC × 10⁶ | Spleen WT (gr) | Leukemia |
| 1 BM (3) | 37 | 20 | 0.70 | Yes |
| 2 Spleen (3) | 27 | 7 | 0.54 | Yes |
| 3 Spleen (4) | 33 | N/A | 0.78 | Yes |
| 4 BM (4) | 37 | 15 | 0.47 | Yes |
| 5 BM (5) | 47 | 17 | 0.61 | Yes |

(3), (4), (5), indicate the cells from donor mouse in table 1. 1 mouse died before analysis.
N/A = not assessed.

### Example 5

### Elevated Trib2 Expression is Found in Human M2 and M4 AML

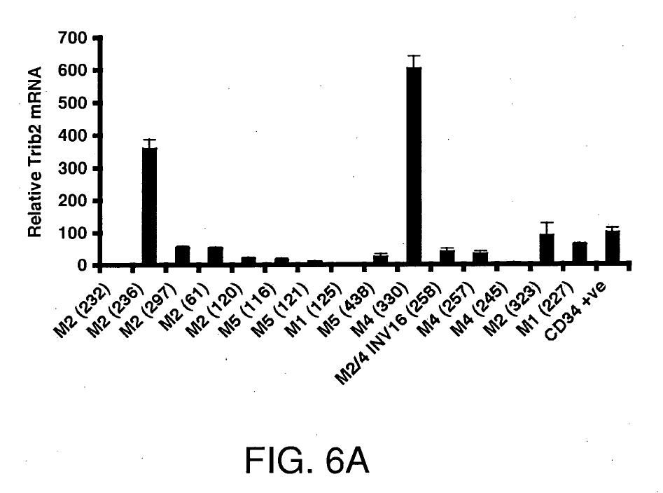

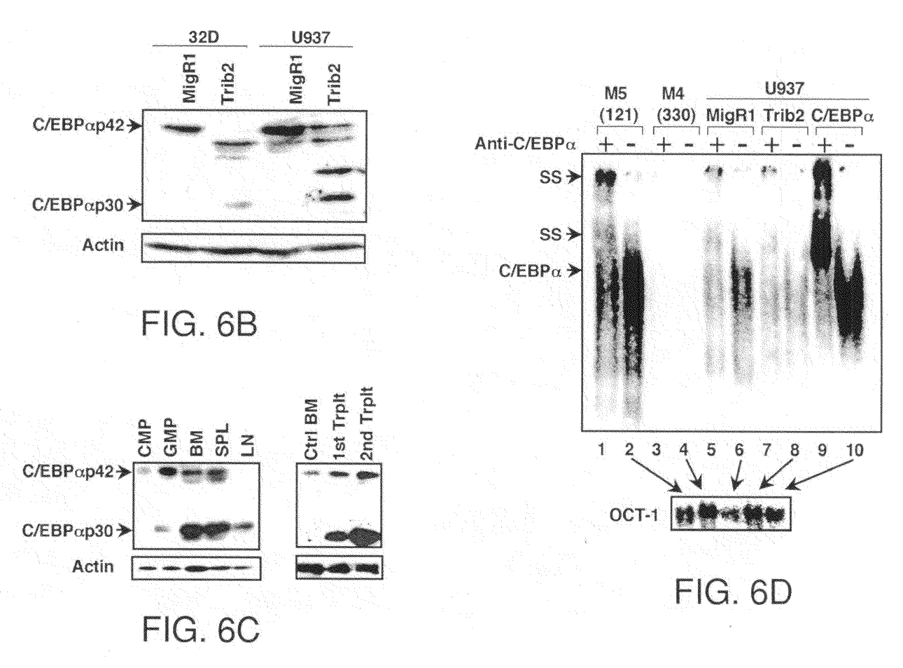

Trib2-induced AML is phenotypically and histologically similar to human M2 and M4 AML. Therefore, mRNA expression level was assessed in a variety of human AML subtypes. Real-time RT-PCR was performed using human specific Trib2 primers on human cDNA samples and compared to the level of Trib2 mRNA expression found in normal CD34 positive cells. In a panel of 15 samples containing M1, M2, M4 and M5 subtypes, 2 samples were found to express significantly elevated levels of Trib2 mRNA. >3 fold higher expression was found in M2-AML sample (˜60% blasts), and >5 fold higher Trib2 expression found in M4-AML sample (>95% blasts) (FIG. 6A). These human M2 and M4 samples did not have any known cytogenetic abnormalities. This correlation of Trib2 mRNA expression with M2 and M4 AML subtypes appears to be significant as 2/15 human samples analyzed expressed elevated levels of Trib2.

### Example 6

### Trib2 Expression Reduces Wild Type C/EBPα Expression and Increases the Dominant Negative C/EBPαp30, and Inhibits DNA Binding Activity

C/EBPα mutations have been found to be exclusively associated with human AML and subtypes M1, M2, and M4 reviewed in (Leroy et al., Leukemia 19:329-334 (2005)). These mutations can lead to decreased wild type C/EBPαp42 expression and increased C/EBPαp30 (dominant negative) expression. C/EBPαp42 is also a critical transcription factor in granulocytic differentiation (Zhang et al., supra, 1997). In addition, the degradation of Slbo (the Drosophila C/EBP homolog) by Drosophila tribbles has been reported (Rorth et al., Mol. Cell 6:23-30 (2000)). As the data set forth elsewhere herein has shown, there is a correlation between Trib2 and AML, therefore with human AML M2 and M4 subtypes, the mechanistic function of Trib2 in AML and whether it affects C/EBPα protein was further investigated.

To address whether Trib2 altered C/EBPα protein expression, 32D and U937 cells were transduced with MigR1 and Trib2. At 48 hours cells were sorted for GFP expression, protein extracts were taken and subjected to western blotting for C/EBPα expression. In both cell lines, reduction of C/EBPαp42 full-length protein was detected. Furthermore, an increase in the C/EBPαp30, the dominant negative protein in Trib2 cell extracts, was detected (FIG. 6B).

To determine if this effect occurs in Trib2-induced AML, protein extracts were taken from bone marrow, spleen and lymph nodes of primary and secondary leukemic mice. Indeed, decreased C/EBPαp42 expression and increased C/EBPαp30 expression was found in all samples (FIG. 6C, left panel). Importantly, the ratio of C/EBPαp42 to C/EBPαp30 proteins was lower in primary leukemic samples and further reduced in secondary leukemic mice (FIG. 6C, right panel). As can be seen in FIG. 6C (left panel), C/EBPαp42 levels in normal hematopoiesis increase at the CMP to the GMP stage, and the ratio of C/EBPαp42 to C/EBPαp30 is greater than 1. If the ratio of C/EBPαp42 to C/EBPαp30 is lower than 1, then C/EBPαp30 acts as a dominant negative to C/EBPαp42 and inhibits its function (Calkhoven et al., Genes Dev. 14:1920-1932 (2000)). Thus, Trib2 appears to promote production of C/EBPαp30 dominant negative protein and may explain the decreased granulopoiesis and increased progenitor proliferation seen in vivo leading to AML in Trib2-induced AML.

To address if the effect of Trib2 on C/EBPαp42 led to an inhibition of its DNA binding function, a key function of C/EBPαp42 granulocytic differentiation activity (Keeshan et al., supra, 2003; Wang et al., Oncogene 22:2548-2557 (2003)), nuclear extracts from MigR1, Trib2, and C/EBPα transduced U937 cells were tested for DNA binding activity of C/EBPα cDNA probe with a consensus C/EBP site in the human G-CSF receptor promoter was used in EMSA. The positive control, U937 cells transduced with C/EBPα (FIG. 6D, lane 10) indicates that the C/EBPα protein complex can be supershifted with a C/EBPα specific antibody (FIG. 6D, lane 9). The C/EBPα complex is much reduced in cells expressing Trib2 (FIG. 6D, lane 8) compared to MigR1 (FIG. 6D, lane 6), as is the supershift complex (FIG. 6D, lanes 5 and 7). These data demonstrate that Trib2 expression inhibits the DNA binding function of C/EBPαp42.

To address whether human samples that had elevated levels of Trib2 shown in FIG. 6A, also displayed inhibition of C/EBPαp42 DNA binding activity, nuclear extracts from M4-AML with elevated Trib2 expression were compared to samples with low levels of Trib2 expression and subjected to EMSA as described above. Significant C/EBPαp42 DNA binding activity was detected in the sample with low levels of Trib2, compared to the human sample with elevated Trib2 expression that exhibits no C/EBPαp42 DNA binding activity (FIG. 6D, lanes 1-4). Integrity and levels of DNA binding proteins in these samples were comparable as shown by OCT-1 (FIG. 6D, lower panel). These data demonstrate that elevated Trib2 expression in human AML corresponds with low C/EBPα expression and activity, as shown by reduced C/EBPα complex.

### Example 7

### Trib2 Binds and Degrades C/EBPαp42 via the Proteasome and Inhibits its Functional Activity

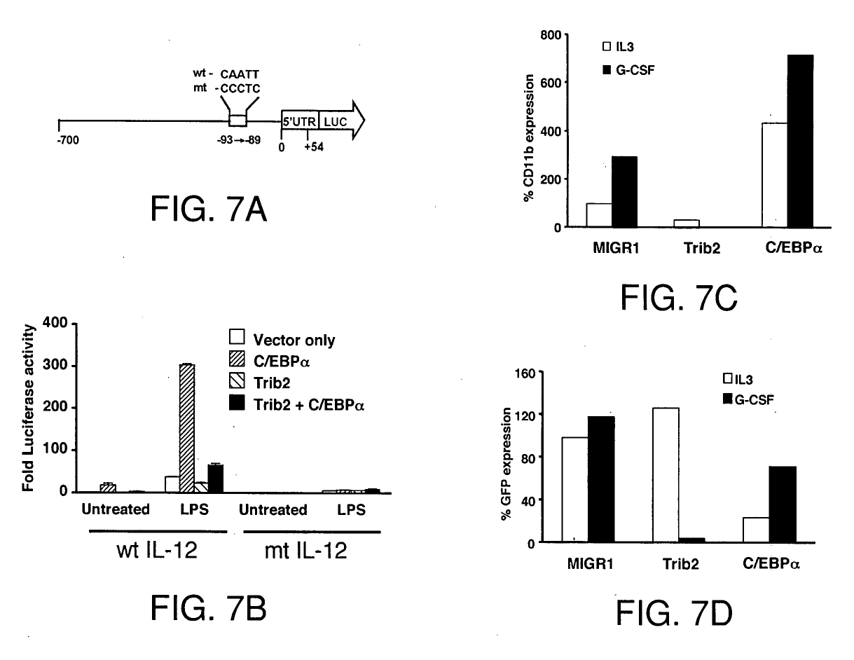

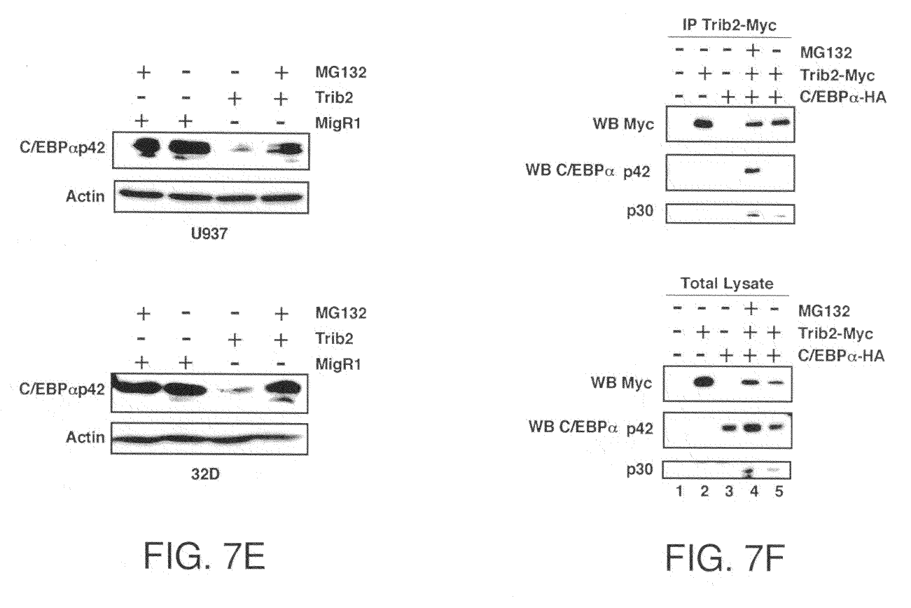

C/EBPαp42 can function by protein-protein hetero-or homodimer interactions, and while Trib2 inhibited its DNA binding activity, it was investigated whether this and the effect on C/EBPαp42 and C/EBPαp30 expression levels led to inhibition of its functional activity in vivo. To address this, transcriptional activity was assessed in RAW macrophage cells that respond to LPS treatment. The IL-12 promoter contains a C/EBP consensus binding site that is required for the induction of IL-12 transcription as shown in the schematic in FIG. 7A (Plevy et al., Mol. Cell Biol. 17:4572-4588 (1997)). RAW cells were transfected with Trib2 and C/EBPα alone, or co-transfected with both Trib2 and C/EBPα and a wild type IL-12 promoter luciferase reporter construct or a IL-12 promoter construct containing a mutated C/EBPbinding site. After 24 hours, cells were treated with LPS (100 ng/ml) for 8 hours and luciferase activity was measured. Reporter luciferase activity for each sample was normalized to the Renilla luciferase activity for the same sample. In the absence of LPS, C/EBPαp42 increased IL-12 promoter luciferase activity that was blocked when Trib2 was co-expressed. In response to LPS, induction of IL-12 reporter activity was enhanced, which could be significantly blocked by co-expression of Trib2 (FIG. 7B).

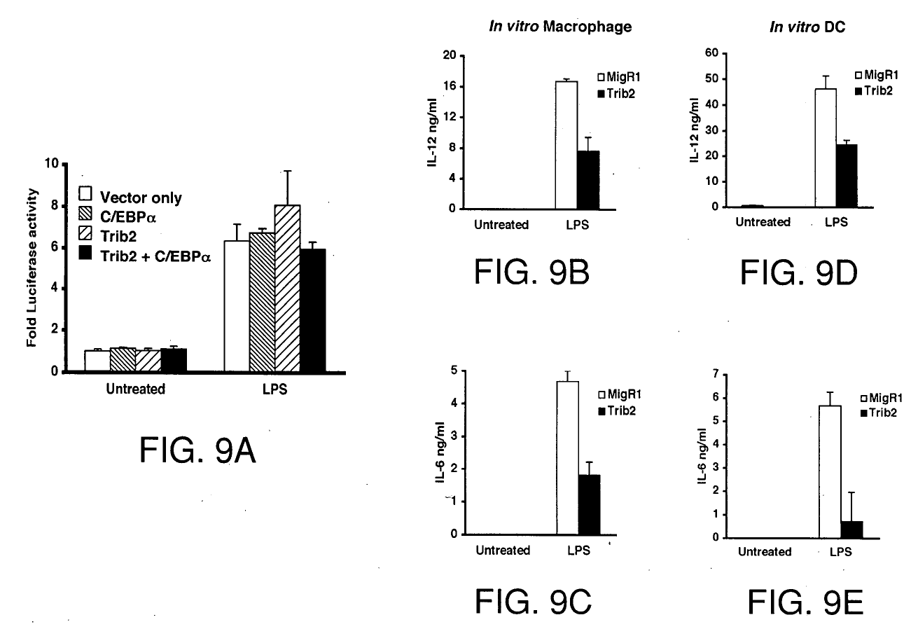

The effect of Trib2 on C/EBPαp42-induced IL-12 reporter activity was specific to C/EBP, as no effect was seen on the IL-12 induction by LPS when C/EBP mutant luciferase construct was co-transfected with C/EBPαp42 and Trib2 (FIG. 7B). Specificity of Trib2-mediated C/EBPα inhibition was further confirmed using a luciferase NFκB consensus reporter construct where no effect was seen. In addition to IL-12, IL-6 contains a C/EBP binding site in its promoter. BM-DCs and macrophages from Trib2 chimeric mice (FIGS. 8C-8E) produced less IL-12 and IL-6 cytokines into the supernatants after treatment with LPS compared to MigR1 control cultures (FIGS. 9B-9E). These data confirm the inhibition of C/EBPα transcriptional function by Trib2 protein expression.

An inhibition of granulopoiesis was observed in vivo in Trib2 chimeric mice, and to address if this was specifically due to Trib2-dependent inhibition of C/EBPαp42 activity, the 32D cell line model that undergoes granulocytic differentiation in response to G-CSF in a C/EBPαp42-dependent manner (Wang et al., Blood 94:560-571 (1999)) was used. 32D cells were transduced with MigR1, Trib2 and C/EBPαp42 (as a positive control) in the presence of IL-3 or G-CSF. CD11b expression was increased in response to G-CSF in 32D-MigR1 cells indicative of granulocytic differentiation (morphological features of neutrophilic differentiation were confirmed by cytospin) and in 32D-C/EBPαp42 cells in both IL-3 and G-CSF. 32D-Trib2 cells however did not differentiate in response to G-CSF, and CD11b expression was reduced in these cells in IL-3 when compared to 32D-MigR1 cells (FIG. 7C). GFP expression was also monitored in these conditions to ascertain whether 32D cells could maintain overexpression of Trib2. 32D-Trib2 cells were maintained and proliferated in IL-3 conditions, however expression of Trib2 (GFP) was lost in G-CSF conditions (FIG. 7D). When 32D cells were sorted for GFP after transduction and cultured in G-CSF, the cells died. Therefore, the reduction in GFP expression in FIG. 7D is not due to untransduced cells proliferating in the culture masking the Trib2 effect.

Because Trib2 expression decreases the expression of wild type C/EBPαp42 (FIGS. 6B and 6C), it was investigated whether this was due to proteasomal degradation of the protein. 32D and U937 cells were transduced and sorted for MigR1 and Trib2 expression. Transduced cells were treated with the proteasomal inhibitor MG132 for 2 hours and C/EBPαp42 expression was assayed by western blotting. In both cell lines, C/EBPαp42 expression was restored by pretreatment with MG132 (FIG. 7E). These data demonstrate that Trib2 promotes the proteasomal degradation of C/EBPαp42.

Co-immunoprecipitation was conducted to determine if this effect was a result of Trib2 binding with C/EBPαp42. Binding of Trib2 to C/EBPαp42 could not be detected, however C/EBPαp30 did co-immunoprecipitate in 293T cells co-transfected with myc-tagged (C-terminus) Trib2 and HA-tagged (C-terminus) C/EBPαp42 (FIG. 7F, lane 5). Importantly, when the cells were pretreated with MG132, C/EBPαp42 and Trib2 were detected in co-immunoprecipitates, and HA-C/EBPαp30 binding was also detected (FIG. F, lane 4). These data illustrate that Trib2 binds to C/EBPαp42 and promotes its degradation via the proteasome and increases production of C/EBPαp30.

Taken together, the findings demonstrate Trib2 as a novel gene involved in AML, and increased Trib2 expression correlates with M2 and M4 human AML subtypes. These data also explain mechanistically how Trib2 expression can promote AML, through the deregulation of an important transcription factor of myeloid development, C/EBPα.

The disclosures of each patent, patent application and publication cited or described in this document are hereby incorporated herein by reference, in their entirety. However, the disclosed dates of publication may be different from the actual publication dates, which may need to be independently confirmed. No reference identified herein is to be construed as an admission that the present invention is not entitled to antedate such publication by virtue of prior invention.

While the foregoing specification has been described with regard to certain preferred embodiments, and many details have been set forth for the purpose of illustration, it will be apparent to those skilled in the art, that without departing from the spirit and scope of the invention, the invention may be subject to various modifications and additional embodiments, and that certain of the details described herein can be varied considerably without departing from the basic principles of the invention. Such modifications and additional embodiments are also intended to fall within the scope and spirit of the invention appended claims.

1-20. (canceled)

21. A method of diagnosing Acute Myeloid Leukemia (AML) in a patient, the method comprising the steps of:
obtaining a myeloid cell from the patient;
assessing Trib2 levels in the myeloid cell;
comparing the assessed level of Trib2 in the patient's myeloid cell to Trib2 levels in a myeloid cell obtained from a healthy control subject; and
determining whether there is a measurable increase of Trib2 indicative of AML in the patient's cell, as compared with the level for the healthy control subject.

22. The method of claim 21, wherein assessing Trib2 comprises assessing Trib2 mRNA levels.

23. The method of claim 21, wherein assessing Trib2 comprises assessing Trib2 polypeptide.

24. The method of claim 23, wherein assessing Trib2 polypeptide comprises contacting the Trib2 polypeptide with an antibody thereto.

25. The method of claim 21, wherein the AML is either M4-AML or M5-AML.

26. A method of diagnosing AML in a patient, the method comprising the steps of:
obtaining a myeloid cell from said patient;
assessing C/EBPαp30 levels in the myeloid cell;
comparing the assessed level of C/EBPαp30 in the patient's myeloid cell to C/EBPαp30 in a myeloid cell obtained from a healthy control subject; and
determining whether there is a measurable increase of C/EBPαp30 indicative of AML in the patient's cell, as compared with the level for the healthy control subject.

27. The method of claim 25, further comprising assessing C/EBPαp42 levels in the patient's myeloid cell and comparing that level to C/EBPαp42 levels in a myeloid cell of the healthy control subject, wherein a measurable decrease of C/EBPαp42 in the patient, when compared with the level of C/EBPαp42 in the myeloid cell of the healthy control subject, is further indicative of a diagnosis of AML in the patient,

28. The method of claim 26, wherein assessing C/EBPαp30 comprises assessing C/EBPαp30 mRNA levels.

29. The method of any one of claim 26, wherein assessing C/EBPαp30 comprises assessing C/EBPαp30 polypeptide levels.

30. The method of claim 26, wherein the AML is either M2-AML or M4-AML.

31. A method of inducing maturation in vivo, in vitro or ex vivo, of a monocyte from a myeloid cell, the method comprising administering Trib2 polynucleotide or a Trib2 polypeptide to the myeloid cell.

32. The method of claim 31, wherein Trib2 polypeptide is expressed from the Trib2 polynucleotide administered to the myeloid cell.

33. A method of treating a patient having AML, the method comprising administering to the patient a Trib2 inhibitor.

34. The method of claim 33, wherein the Trib2 inhibitor comprises either an inhibitor of Trib2 polypeptide or an inhibitor of Trib2 polynucleotide expression.

35. The method of claims 33, further comprising selecting the Trib2 polypeptide inhibitor from either a polypeptide that binds to a Trib2 polypeptide or to a C/EBPαp30 polypeptide.

36. The method of claim 35, further comprising selecting the Trib2 polypeptide inhibitor from an antibody to a Tribe2 polypeptide, or to either a Trib2 antisense or RNAi composition.

37. The method of claim 34, further comprising selecting the inhibitor of Trib2 polynucleotide expression from the group consisting of Trib2RNA-binding protein, Trib2 DNA-binding protein, Trib2 antisense composition and Trib2 RNAi polynucleotide.

38. A method of diagnosing a malignancy associated with Trib2, C/EBPαp30 or C/EBPαp42 in a patient, the method comprising the steps of:
obtaining a hematopoietic stem cell from the patient;
assessing the level of Trib2, C/EBPαp30 or C/EBPαp42, respectively in the hematopoietic stem cell;
comparing the assessed level of Trib2, C/EBPαp30 or C/EBPαp42, respectively to a level of Trib2, C/EBPαp30 or C/EBPαp42, respectively, from a hematopoietic cell obtained from a healthy control subject; and
determining whether there is a measurable increase of Trib2, C/EBPαp30 or C/EBPαp42, respectively, in the patient's cell, indicative of malignancy in the patient, as compared with the level for the healthy control subject.

39. The method of claim 38, wherein the malignancy is selected from the group consisting of AML and lung cancer.

40. The method of claim 38, wherein the hematopoietic stem cell is a myeloid cell.
